(19) United States
(12) Patent Application Publication
(10) Pub. No.: US 2021/0196206 A1
(43) Pub. Date: Jul. 1, 2021

(54) SYSTEMS AND METHODS FOR SEPSIS RISK EVALUATION

(71) Applicant: DexCom, Inc., San Diego, CA (US)

(72) Inventors: Devon M. Headen, Atlanta, GA (US); Peter C. Simpson, Cardiff, CA (US); Matthew Lawrence Johnson, Solana Beach, CA (US)

(21) Appl. No.: 17/132,855

(22) Filed: Dec. 23, 2020

Related U.S. Application Data
(60) Provisional application No. 62/953,807, filed on Dec. 26, 2019.
(60) Provisional application No. 62/956,044, filed on Dec. 31, 2019.

(51) Int. Cl. A61B 5/00; A61B 5/145; A61B 5/01; A61B 5/0205; G16H 40/67; G16H 50/30; G16H 10/40

(52) U.S. Cl. CPC A61B 5/7275; A61B 5/14546; A61B 5/746; A61B 5/01; A61B 5/0205; A61B 5/024; A61B 5/14503; G16H 40/67; G16H 50/30; G16H 10/40; A61B 2505/05; A61B 5/4866

(57) ABSTRACT

Certain aspects of the present disclosure relate generally to a method for identifying a risk of sepsis in a body of a patient. The method includes measuring lactate concentrations associated with the body over one or more time periods. The method further includes identifying the risk of sepsis based on the lactate concentrations.

### INCORPORATION BY REFERENCE TO ANY PRIORITY APPLICATIONS

This application claims the benefit of U.S. Provisional Application Ser. No. 62/953,807 entitled “SYSTEMS AND METHODS FOR SEPSIS RISK EVALUATION,” which was filed on Dec. 26, 2019, as well as U.S. Provisional Application Ser. No. 62/956,044 entitled “SYSTEMS AND METHODS FOR USING LACTATE SENSING AS A PHYSICAL FITNESS TRAINING AID,” which was filed on Dec. 31, 2019. The aforementioned applications are herein incorporated by reference in their entirety. Any and all applications for which a foreign or domestic priority claim is identified in the Application Data Sheet as filed with the present application are hereby incorporated by reference under 37 CFR 1.57.

### BACKGROUND

Sepsis is a major cause of mortality. There are more than 1.5 million cases of sepsis each year, killing more than 250,000 people in the US alone. Globally, more than 149 million sepsis cases are reported on a yearly basis, with around 11 million deaths. Sepsis may arise as a result of a variety of diseases and conditions, including post-operative infections, urinary tract infections, pneumonia, diarrheal diseases, etc. Generally, multiple factors are involved in infections that lead to sepsis, making it difficult to predict whether a patient will or will not develop sepsis. In addition, diagnosis of sepsis is difficult, with the symptoms being potentially related to or masked by other illnesses or surgical complications. This is especially problematic because early recognition and appropriate antibiotic treatment is of critical importance in minimizing the severity and progression of sepsis.

Elevated blood lactate levels are an important criteria in establishing a sepsis diagnosis. Lactate concentration determination and monitoring are regularly performed in hospitals as a data point for patient care with respect to sepsis development and sepsis recovery evaluation as well as for a variety of other illnesses and conditions.

Lactate testing for this purpose is typically done by drawing blood from the patient and testing the blood for a variety of analytes including lactate with a bench-top blood gas analyzer in a laboratory. However, there are a number of drawbacks associated with the conventional periodic lactate testing through blood draws, which can include the use of finger sticks. First, there is typically a delay associated with obtaining lactate concentration information from blood such that, due to this delay, any lactate concentration measurements derived from a patient's blood may not be representative of the patient's real-time lactate concentration levels. Second, because periodic blood draws are generally performed no more than every 1-12 hours, they provide a limited set of lactate concentration data points, thereby, making it more difficult to establish trends and determine whether a patient is responding to treatment in real-time.

In addition to performing lactate testing for sepsis risk evaluation, in certain cases, lactate testing may be performed for professional athletes to determine their lactate thresholds. For example, during strenuous physical activity, muscles can become deprived of sufficient oxygen to use the normal metabolic pathway. In these cases, the muscle tissue will switch to an anaerobic metabolic pathway that produces lactate. In certain instances, athletic performance may be correlated to the amount of work the muscles can do before switching to the anaerobic metabolic pathway. The greater the work that can be performed prior to the switch, the better the athlete is able to perform. To determine lactate threshold, an athlete will get on a treadmill or exercise bicycle and be subjected to incrementally increased work load. Blood is periodically drawn during the test and the lactate concentration is measured. There will typically be a work load where lactate concentrations start to increase at a high rate. Successful training regimens increase this threshold, and the threshold forms a data point in a fitness evaluation. These tests are used for professional athletes but are expensive and difficult to obtain for people interested in fitness and fitness measures who are not professional athletes.

It should be noted that this Background is not intended to be an aid in determining the scope of the claimed subject matter nor be viewed as limiting the claimed subject matter to implementations that solve any or all of the disadvantages or problems presented above. The discussion of any technology, documents, or references in this Background section should not be interpreted as an admission that the material described is prior art to any of the subject matter claimed herein.

### SUMMARY

In certain embodiments, a method of sepsis risk monitoring comprises entering a health care facility, implanting a sensor system, undergoing a surgical procedure in the health care facility, and leaving the healthcare facility after performance of the surgical procedure with the lactate sensor remaining implanted. The lactate sensor may remain implanted for at least three days after leaving the healthcare facility.

In certain embodiments, a sensor system comprises an implantable lactate sensor, a body temperature sensor, and sensor electronics operably connected to the lactate sensor and the body temperature sensor. In such embodiments, the sensor electronics may be configured to integrate sensor data from the lactate sensor and sensor data from the body temperature sensor to generate a value representative of sepsis risk. Heart rate and respiration rate sensors may also be included as part of the system.

In certain embodiments, an electrochemical lactate sensor comprises two or more electrodes and a sensing membrane overlaying at least a portion of at least one of the two or more electrodes. The sensing membrane comprises an enzyme portion (e.g., comprising lactate oxidase) and a resistance portion that is more permeable to oxygen than lactate.

In certain embodiments, a method of sepsis risk monitoring comprises implanting a sensor system in a patient in the time period between one day before beginning a surgical procedure on a patient and one day after ending the surgical procedure on the patient and leaving the lactate sensor implanted for at least three days after ending the surgical procedure.

In certain embodiments, a method of sepsis risk monitoring comprises selecting a patient for sepsis monitoring, implanting a sensor system in the patient, and performing a surgical procedure on the patient (in either order). The method further comprises discharging the patient following the surgical procedure with the lactate sensor remaining implanted. In certain embodiments, a method of monitoring for post-operative sepsis risk comprises implanting a sensor system within one day of ending a surgical procedure performed in a healthcare facility. The implantation may occur after discharge.

In certain embodiments, a method is provided for identifying a risk of sepsis in a body of a patient. The method includes measuring, using a lactate sensor system including a lactate sensor worn by the patient, lactate concentrations associated with the body over one or more time periods. The method further includes identifying, using the lactate monitoring system, the risk of sepsis based on the lactate concentrations.

In one implementation, a method of activity monitoring comprises implanting a transcutaneous lactate sensor, leaving the transcutaneous lactate sensor implanted for the duration of a sensor session, performing one or more elements of a fitness routine during the sensor session, continuously measuring lactate concentration with the transcutaneous lactate sensor during the sensor session, and storing at least some lactate concentrations measured by the transcutaneous lactate sensor during the sensor session.

In another implementation, a method of activity monitoring comprises placing a first lactate sensor on a subject, leaving the first lactate sensor implanted for the duration of a first sensor session, performing one or more elements of a first fitness routine during the first sensor session, continuously measuring lactate concentration with the first lactate sensor during the first sensor session, and storing at least some first lactate concentrations measured by the lactate sensor during the first sensor session. The first lactate sensor is then removed. The method continues with placing a second lactate sensor on the subject after removing the first lactate sensor, leaving the second lactate sensor implanted for the duration of a second sensor session, performing one or more elements of a second fitness routine during the second sensor session, continuously measuring lactate concentration with the second lactate sensor during the second sensor session, and storing at least some second lactate concentrations measured by the second lactate sensor during the sensor session.

In another implementation, an activity monitoring system comprises a lactate sensor, sensor electronics operably connected to the lactate sensor, a memory operably connected to the sensor electronics for storing measured lactate concentrations, and a processor configured to generate an estimate of aggregate lactate (e.g., estimate of an aggregate of high concentration of lactate developed in the body) over a period of time based at least in part on stored measured lactate concentrations.

In another implementation, an activity monitoring system comprises a lactate sensor, sensor electronics operably connected to the lactate sensor, a memory operably connected to the sensor electronics for storing measured lactate concentrations, and a processor configured to generate an estimate of aggregate lactate over a period of time based at least in part on stored measured lactate concentrations.

In another implementation, a method of activity monitoring comprises placing a lactate sensor on a subject, leaving the lactate sensor on the subject for the duration of a sensor session, performing a plurality of elements of a fitness routine during the sensor session, continuously measuring lactate concentration with the lactate sensor during the sensor session, storing at least some lactate concentrations measured by the lactate sensor during the sensor session, and processing a plurality of lactate concentrations measured by the lactate sensor to generate an estimate of aggregate lactate over a period of time. The lactate sensor may be transcutaneous or non-invasive.

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

Various embodiments are discussed in detail in conjunction with the Figures described below, with an emphasis on highlighting the advantageous features. These embodiments are for illustrative purposes only and any scale that may be illustrated therein does not limit the scope of the technology disclosed. These drawings include the following figures, in which like numerals indicate like parts.

FIG. 1 illustrates an example health monitoring system including a lactate sensor system as well as a mobile computing device, in accordance with certain aspects.

FIG. 2 is a flowchart of a method of monitoring for sepsis risk with a sensor system, in accordance with certain aspects.

FIG. 3 is a flowchart of another a method of monitoring for sepsis risk with a sensor system, in accordance with certain aspects.

FIG. 4 is a flowchart of yet another a method of monitoring for sepsis risk with a health monitoring system, including a sensor system, in accordance with certain aspects.

FIGS. 5A and 5B illustrate an example of a lactate sensor, in accordance with certain aspects.

FIGS. 6A, 6B, and 6C illustrate an example of a sensor system including both a lactate sensor, and associated sensor electronics, in accordance with certain aspects.

FIG. 7 is a block diagram of an example embodiment of sensor electronics, in accordance with certain aspects.

FIG. 8 is a block diagram depicting a computing device configured to perform one or more operations of FIG. 4, in accordance with certain aspects.

FIG. 9 shows a typical determination of “lactate threshold” for an athlete.

FIG. 10 shows lactate levels and heart rate measured for a subject over about a two-hour resistance training workout.

FIG. 11 illustrates an example of using a sensor system as a fitness training aid, in accordance with certain aspects.

FIG. 12 shows an exemplary sensor system, where a lactate sensor communicates with sensor electronics, in accordance with certain aspects.

FIG. 13 illustrates an example of a method of using lactate sensing as a fitness training aid, in accordance with certain aspects.

### DETAILED DESCRIPTION

The following description and examples illustrate some exemplary implementations, embodiments, and arrangements of the disclosed invention in detail. Those of skill in the art will recognize that there are numerous variations and modifications of this invention that are encompassed by its scope. Accordingly, the description of a certain example embodiment should not be deemed to limit the scope of the present invention. To facilitate an understanding of the various embodiments described herein, a number of terms are defined below.

### Definitions

Surgical procedure—A medical procedure that includes, at least in part, physician access to internal physiological structures of a subject with tools and/or instruments.

Fitness routine—A sequence of physical activities planned at least in part in advance and designed to improve one or more bodily functions related to the cardiovascular system, the respiratory system, and/or the muscular system. For example, a series of workouts scheduled to be performed at different times over a period of time, usually several days or weeks.

Element of a fitness routine—A substantially continuous physical activity or a substantially contiguous series of physical activities performed as part of a fitness routine. For example, a given individual workout. Different elements of a single fitness routine are separated in time by a cardiovascular recovery interval such that tissue oxygenation has substantially returned to normal resting levels. For example, going for a 30-minute run on one day and lifting weights at the gym for an hour on the next day would constitute two different elements of a single fitness routine.

Monitor—A device for measuring a physiological parameter of a subject such as but not limited to one or more of heart rate, temperature, and blood analyte concentrations. A monitor may be comprised of a plurality of operably connected or connectable components. Each such cooperating component is individually a monitor, as well as any combination thereof.

Healthcare facility monitor—A monitor that under normal use is used inside a health care facility and is not taken out of a health care facility by a subject with which the monitor was used.

Temporary monitor—A monitor that is intended for a single use by a single subject over a defined duration (e.g., of not more than 90 days).

Binary output—A monitor output that categorizes a monitored subject as either having a specified condition or not having the specified condition.

Monitor binary sensitivity—The probability that during use a binary output of a given monitor will correctly categorize a subject with the condition as having the condition. Monitor binary sensitivity may be referred to as simply sensitivity, where the meaning will be clear from context.

Monitor binary specificity—The probability that during use a binary output of a given monitor will correctly categorize a subject without the condition as not having the condition. Monitor binary specificity may be referred to as simply sensitivity, where the meaning will be clear from context.

Sensor—The component or region of a monitor by which a physiological, environmental, or other parameter can be quantified, including but not limited to the implanted portion of an analyte monitor, an internal or external temperature sensor, a pressure sensor, a motion sensor, or a sensor of any other parameter.

Lactate—Includes one or both the L and D enantiomers of the molecule individually and any combination thereof. In addition to the ion/salt, the term lactate as used herein includes lactic acid. Typically, the L-lactate ion is measured in vivo.

Lactate Sensor—A structure incorporating any mechanism (e.g., enzymatic or non-enzymatic) by which an amount or concentration of lactate can be quantified. For example, some embodiments utilize a membrane that contains lactate oxidase that catalyzes the conversion of oxygen and lactate to hydrogen peroxide and pyruvate. Using this reaction, an electrode can be used to monitor the current change in either the co- reactant or the product to measure lactate concentration. Lactate dehydrogenase is another suitable catalyst.

Body temperature—may include, among other types of body temperatures, core body temperature of internal organs. Rectal and vaginal temperature measurements are generally the closest to actual core body temperature. Measurements in other locations such as the mouth or skin can be calibrated to provide suitable estimates for use by the lactate monitors described herein.

Operably connected—One or more components of a device or system being linked to another component(s) of the device or system in a manner that allows transmission of signals between the components. For example, one or more electrodes can be used to detect the amount of lactate in a sample and convert that information into a signal, e.g., an electrical or electromagnetic signal; the signal can then be transmitted to an electronic circuit. In this case, the electrode is operably connected to the electronic circuitry. The term operably connected includes signal transmission or exchange without physical contact, e.g., wireless connectivity.

Determining—Calculating, computing, processing, deriving, investigating, looking up (e.g., looking up in a table, a database or another data structure), ascertaining, estimating, detecting, and the like. Also, “determining” may include receiving (e.g., receiving information), accessing (e.g., accessing data in a memory) and the like. Also, “determining” may include resolving, selecting, choosing, calculating, deriving, establishing and/or the like. Determining also includes classifying a parameter or condition as present or not present, and/or meets a predetermined criterion, including that a threshold has been met, passed, exceeded, and so on.

Substantially—Largely but not necessarily wholly that which is specified such that at least most of the practical effect or purpose of that which is specified is maintained.

Continuous Monitor—A monitor that is configured to periodically measure a physical or biological parameter at a certain frequency. This includes signal sampling at any interval appropriate to the measurement signal, ranging from fractions of a second up to, for example, 1, 2, or 5 minutes, or longer. For in vivo analyte sensing, taking a sample every 1-30 minutes is typically more than sufficient to be within the meaning of the term continuous. Independent of sampling rate considerations, for monitors that are in use in a sensor session lasting more than one day, the term continuous can include gaps in data acquisition totaling less than half of the sensor session. It will be appreciated that although such gaps occur for a variety of reasons related to monitor operation, they are usually incidental to the monitoring process, and typically total less than 20%, less than 10%, or less than 5% of the duration of a sensor session.

Sensing Membrane—One or more layers of material on or over a substrate that includes one or more functional domains or regions that in combination provide measurement functionality to a sensor.

Sensor data—Any information associated with one or more sensors. Sensor data includes a raw data stream, or simply data stream, of analog or digital signals directly related to a measured analyte from an analyte sensor (or other signal received from another sensor), as well as calibrated and/or filtered raw data. In one example, the sensor data comprises digital data in “counts” converted by an A/D converter from an analog signal (e.g., voltage or amps) and includes one or more data points representative of an analyte concentration (e.g., a lactate concentration). Thus, the terms “sensor data point” and “data point” refer generally to a digital representation of sensor data at a particular time. The terms broadly encompass a plurality of time spaced data points from a sensor which comprises individual measurements taken at time intervals ranging from fractions of a second up to, e.g., 1, 2, or 5 minutes or longer. In another example, the sensor data includes an integrated digital value representative of one or more data points averaged over a time period. Sensor data may include calibrated data, smoothed data, filtered data, transformed data, and/or any other data associated with a sensor.

Sensor electronics—The components (for example, hardware and/or software) of a monitor that are configured to process data. Sensor electronics may be arranged and configured to measure, convert, store, transmit, communicate, and/or retrieve sensor data associated with an analyte sensor.

Sensor sensitivity—The relationship between the magnitude of a sensor measurement signal and the concentration of an analyte being measured by the sensor. Sensor sensitivity may be linear or non-linear. Sensor sensitivity may be referred to as simply sensitivity, where the meaning will be clear from context.

Sensor session—A time duration over which a given sensor makes parameter measurements of a subject. The sensor may be but does not have to be continuously implanted or otherwise attached to the subject over the course of the entire sensor session. For an implantable sensor, a sensor session may be the period of time starting at the time the sensor is implanted to the time the sensor is removed.

Transcutaneous—Located under the epidermis of a subject, including locations in the dermis, hypodermis, and/or underlying muscle tissue, but excluding intravenous or intraarterial locations.

Transcutaneous sensor—A sensor configured for transcutaneous implantation.

App—A software program capable of executing on smartphone operating systems such as iOS and Android. Although an app is generally designed for operation on mobile devices, an app can be executed on non-mobile devices that are running an appropriate operating system.

Server—Processing hardware coupled to a computer network having network resources stored thereon or accessible thereto that is configured with software to respond to client access requests to use or retrieve the network resources stored thereon.

### Sepsis Monitoring and Risk Evaluation

Despite its seriousness as a health care problem, little progress has been made in reducing the occurrence or mortality rates of sepsis, and little progress appears to be on the horizon. In a recent article in the Journal of the American Medical Association (JAMA), a “Key Point” of the study was identified as “sepsis is a leading cause of death in US hospitals, but most of these deaths are unlikely to be preventable through better hospital-based care” (Rhee, et al., Prevalence, Underlying Causes, and Preventability of Sepsis-Associated Mortality in US Acute Care Hospitals, JAMA Network Open 2019 2(2) e187571). Sepsis is also the leading cause of 30-day readmissions after initial discharge, and these sepsis readmissions are on average longer and more expensive than other readmission diagnoses such as heart failure, pneumonia, and COPD. (Mayr et al., JAMA Research Letter, Volume 317, No. 5, Feb. 7, 2017).

Lactate levels are an important component of sepsis diagnosis and evaluation of sepsis treatment efficacy. Systems and methods described herein utilize continuous lactate monitoring to address sepsis diagnosis and treatment in a novel way.

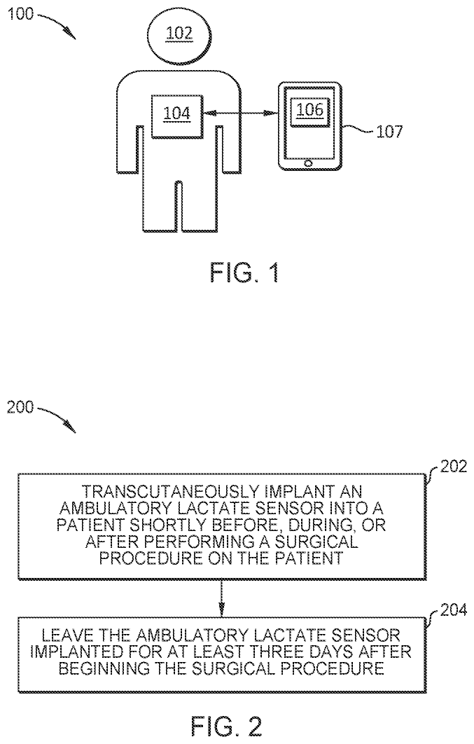

FIG. 1 illustrates an example health monitoring system 100 including lactate sensor system 104 (“sensor system 104”) as well as a mobile computing device 107 configured to execute a health monitoring software application (“health monitoring application”) 106. As shown, sensor system 104 is worn by the patient 102. Sensor system 104 is a wearable or portable sensor system that may be worn by the patient 102 either by implanting (at least partially) the sensor system 104 in the body or non-invasively wearing it.

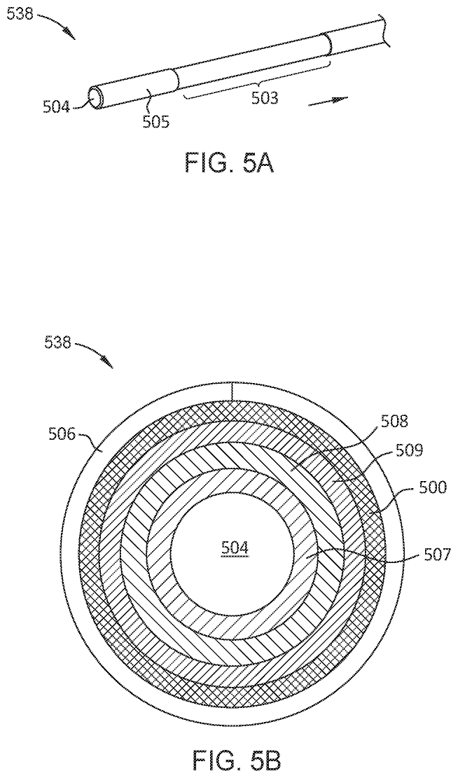

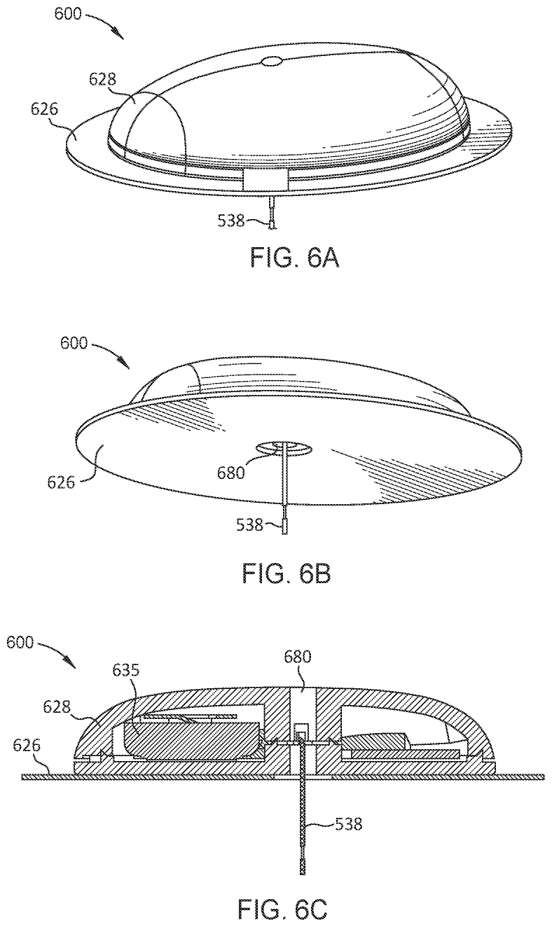

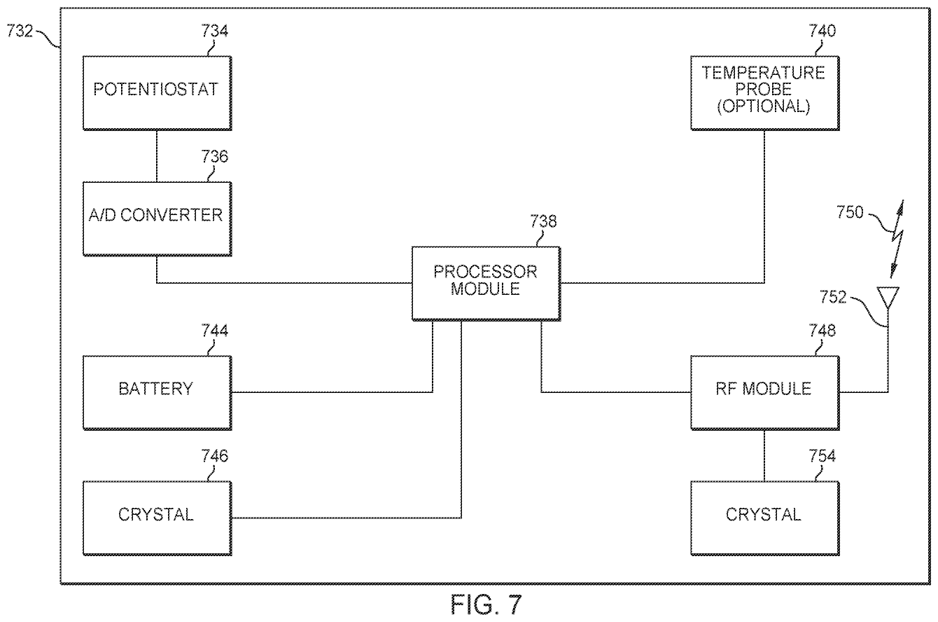

Sensor system 104 comprises a lactate sensor (shown in FIGS. 5-6) as well as sensor electronics (shown in FIG. 7). Sensor system 104 is configured to continuously monitor lactate concentration levels of patient 102 and transmit the resulting lactate concentration measurements to health monitoring application 106. Components of sensor system 104 are described in further detail with respect to FIGS. 5-6. Health monitoring application 106 configures mobile computing device 107 to perform, for example, lactate monitoring for sepsis risk and/or other health related monitoring (e.g., athletic performance monitoring, as described below). Mobile computing device 107 may be operated by patient 102 or another user (e.g., caregiver of patient 102). In addition, although a mobile computing device 107 is shown in FIG. 1, in certain other embodiments, a non-mobile computing device may instead be used.

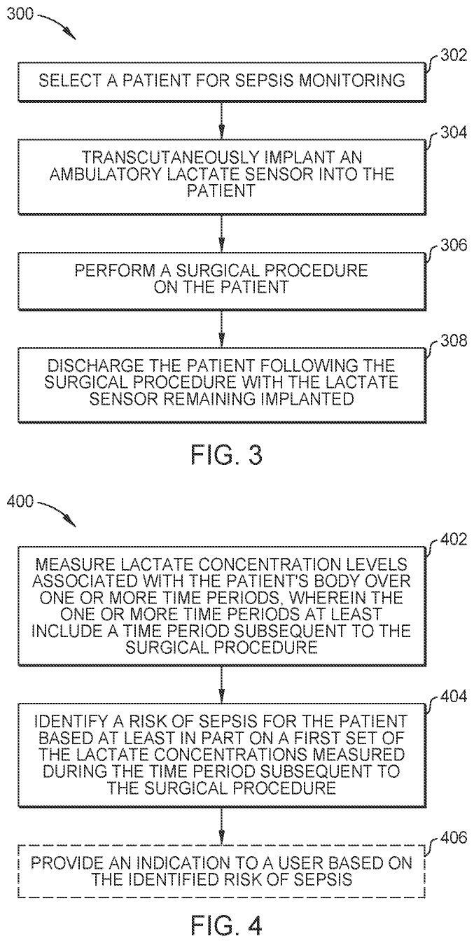

As described above, sepsis may develop as a result of a variety of conditions and diseases, such as post-operative infections, urinary tract infections, pneumonia, diarrheal diseases. The health monitoring system 100 described herein may be used to monitor sepsis risk for a patient with any of the diseases or conditions described above or for any other disease where sepsis risk may exist. FIGS. 2-3 describe various methods of implanting a sensor system (e.g., sensor system 104) in a patient to monitor the patient for post-operative sepsis risk. FIG. 4, more generally, illustrates a method of sepsis risk monitoring for a patient with any disease or condition (e.g., post-operative infections, urinary tract infections, pneumonia, diarrheal diseases, etc.) that may result in sepsis.

Lactate monitoring for sepsis risk can be performed both in the hospital before discharge and at home after discharge with continuous use of the same device. In order to monitor a patient for sepsis risk, a sensor system (e.g., sensor system 104) may be first implanted in the patient. FIGS. 2-3 describe various methods associated with implanting a sensor system in a patient for sepsis risk monitoring. FIG. 2 is a flow chart 200 of a sepsis risk monitoring method. At block 202 a sensor system is implanted in a patient shortly before, during, or shortly after performing a surgical procedure on the patient. The surgery may be an elective or a non-elective surgery. Shortly before or after may be defined as sometime between one day (24 hours) before the surgery begins to one day (24 hours) after the surgery ends. In certain embodiments, shortly before the surgery may be defined as multiple days before the surgery begins. For example, a sensor system may be implanted in a patient any time in the range of 1 to 30 days before the surgery begins.

At block 204, the sensor system remains implanted, for example, for at least 3 days (72 hours) after ending the surgical procedure. The length of time the sensor system remains implanted may be based at least in part on an evaluation of patient recovery from surgery and the associated decrease in the chance that sepsis has or will develop. This may vary from procedure to procedure and patient to patient, and may be, for example, at least 1 day after ending the surgical procedure, at least 3 days after ending the surgical procedure, at least 10 days after ending the surgical procedure, or at least 30 days after ending the surgical procedure, or longer.

As noted briefly above, an important benefit of implanting a lactate sensor is that its use does not need to end with the end of hospitalization (e.g., post-surgical hospitalization). A patient can wear the device at home after discharge where it can continue to provide sepsis risk monitoring for a longer period than is necessary for the hospitalization itself. Another beneficial aspect of this form of lactate level monitoring is that it does not involve a change in healthcare facility standard procedure with respect to lactate level monitoring. Instead, it is a supplement to them.

As a supplement, its use can be at the discretion of the physician based on their own professional judgment with respect to sepsis risks. For example, post-surgical risks are higher with some types of elective surgeries. Surgery on organs of the digestive system are especially problematic. The digestive system is a subset of human internal organs including the esophagus, liver, gallbladder, stomach, spleen, pancreas, small intestine, and large intestine. Surgeries on these organs, especially the esophagus, pancreas, and stomach have been found to result in both more instances and more costly instances of sepsis. Age, over 60 years old for example, is another factor that increases sepsis risk. A physician can therefore select patients with whom to utilize a supplemental sensor system based on the nature of the surgical procedure and/or the age of the patient.

In certain embodiments, the implantation of the sensor is preferably transcutaneous. Transcutaneous analyte sensors have been used with success in continuous glucose monitoring (CGM) applications for diabetics. These on-body devices have become safe, reliable, unobtrusive, and painless. Certain aspects of lactate sensing, as has been determined by the inventors listed in the present application, are similar with respect to the analog and digital components needed to perform the measurements. Thus, the lactate monitors proposed herein may have certain similarities to the glucose monitors currently in widespread use. This may help lower apprehension on the part of patients to wear the lactate monitors after discharge. In fact, the knowledge that a sepsis risk sensor is going to continue to be used after discharge may make many patients more comfortable and confident when leaving the healthcare facility after surgery. It may also be noted that one aspect of continuous analyte sensors that has made their use impractical difficult has been the need for the sensor to stabilize in vivo for an hour or more before data can be acquired. With the particular methods described herein, by the time the patient is discharged, the stabilization time will be long passed, and proper function of the device can be verified prior to discharge.

FIG. 3 illustrates a flow chart 300 of another sepsis risk monitoring method. At block 302, a patient is selected for sepsis monitoring. As discussed above, the patient selection may be based on the nature of the surgery to be performed, the age of the patient, and/or any other factors the physician or healthcare facility deems relevant. At block 304, a sensor system is implanted in the patient. At block 306, a surgical procedure is performed on the patient. At block 308, the patient is discharged following the surgical procedure with the lactate sensor remaining installed. It will be appreciated that although block 304 precedes block 306 in the flowchart of FIG. 3, it would be possible to implant the sensor system either before, during, or after performing the surgical procedure. However, pre-surgery implantation would be convenient and could provide a pre-surgery lactate baseline measurement of the patient, as further described in relation to FIG. 4.

FIG. 4 illustrates a flow chart 400 of a method of sepsis risk monitoring performed by a lactate monitoring system, such as health monitoring system 100. Note that the sepsis risk monitoring method of FIG. 4 may be performed for a patient with any disease or condition (e.g., post-operative infections, urinary tract infections, pneumonia, diarrheal diseases, etc.) that may result in sepsis. Further, note that, for ease of understanding, the blocks of flow chart 400 are described herein as being performed by health monitoring system 100. However, the use of any similar health monitoring system to perform the method of FIG. 4 is also within the scope of this disclosure.

At block 402, sensor system 104 of system 100 measures lactate concentration levels associated with a patient over one or more time periods. In certain embodiments, the one or more time periods include a single continuous time period. In some embodiments, the one or more time periods may be associated with periods for monitoring lactate concentrations for various purposes. In certain embodiments, the single continuous time period may start prior to, during, or subsequent the occurrence of a sepsis risk event (“sepsis event”), which refers to an event (e.g., disease, condition, surgery/operation, etc.), that may expose the patient to the risk of developing sepsis. For example, in certain embodiments, the sensor system 104 may be implanted in the patient when the patient is not exposed to the risk of sepsis yet or is otherwise in a normal physical state. Once implanted, the sensor system 104 begins to continuously measure the patient's lactate concentration levels. At some later point in time, the patient may experience a sepsis event. In certain embodiments, a time period during which sensor system 104 measures lactate concentration levels of the patient prior to the sepsis event may be referred to as a pre-sepsis-event time period. In certain embodiments, a time period during which sensor system 104 measures lactate concentration levels of the patient subsequent to the sepsis event may be referred to as a post-sepsis-event time period. In certain embodiments, the pre-sepsis-event and the post-sepsis-event time periods may be part of a single continuous time period. One example of where the pre-sepsis-event and the post-sepsis-event time periods may be considered to be parts of a single continuous time period, is when there is no disruption in measuring the patient's lactate concentration levels between the two time periods and/or when the time of the sepsis event (e.g., the time of when it starts and/or ends) is not easily identifiable.

In certain other embodiments, the pre-sepsis-event and the post-sepsis-event time periods may be distinct time periods. One example of where the pre-sepsis-event and the post-sepsis-event time periods may be considered as distinct, is when there is a slight disruption in measuring the patient's lactate concentration levels between the two time periods and/or when the time of the sepsis is easily identifiable.

For example, when the patient is monitored for post-operative sepsis risk, the one or more time periods may include a time period prior to the patient's surgery (“pre-surgery time period”) and/or a time period after the patient's surgery (“post-surgery time period”). In this example, the patient's surgery is the sepsis event. The pre-surgery time period (an example of pre-sepsis-event time period) refers to a time period during which sensor system 104 measures lactate concentration levels of the patient prior to the patient's surgery. For example, in certain embodiments, sensor system 104 may be implanted in the patient a number of days or hours prior to the surgery. In certain embodiments, sensor system 104 may be implanted in the patient by a clinician during a visit. In certain other embodiments, sensor system 104 may be implanted in the patient by the patient or the patient's caregiver without the need to visit a health care facility.

In certain embodiments, sensor system 104 may be implanted in the patient at a time that does not fall during the pre-sepsis-event time period. For example, sensor system 104 may be implanted in the patient while the sepsis event is occurring or during the post-sepsis-event time period.

After being implanted, sensor system 104 may automatically, or in response to receiving an indication, begin measuring the patient's lactate concentration levels. In certain embodiments, the indication may be received from health monitoring application 106 that is executing on mobile computing device 107. For example, once the sensor system 104 is implanted, the patient or the patient's caregiver may provide user input to health monitoring application 106 to send an indication to and cause the sensor system 104 to begin measuring the patient's lactate concentration levels. In certain embodiments, the user input received by health monitoring application 106 may cause it to enter sepsis monitoring mode under which health monitoring application 106 may utilize sepsis-specific algorithms to identify sepsis risk. For example, health monitoring application 106 may initially be in a non-sepsis mode, where sepsis related algorithms and techniques are not used to identify sepsis risk (thereby using less compute and memory resources) and then, in response to the user input, transition into the sepsis monitoring mode. In certain embodiments, the user input may indicate the time period during which sensor system 104 is beginning to measure the patient's lactate concentration levels and/or the date of a sepsis event (e.g., date of the surgery or some other disease or condition).

For example, if the sensor system 104 is being implanted in the body pre-surgery, then the user input may indicate a date/time of surgery, which itself indicates that: (1) until the indicated date/time of surgery, any lactate concentration measurements received by health monitoring application 106 are going to correspond to the patient's pre-surgery lactate concentration levels and that (2) subsequent to the indicated date/time of surgery, any lactate concentration measurements received by health monitoring application 106 are going to correspond to the patient's post-surgery lactate concentration levels. As described further below, pre-sepsis-event lactate concentration measurements may be used to personalize sepsis risk identification, which, in certain embodiments, may result in providing more accurate and effective sepsis risk monitoring and analysis (e.g., by reducing false positives). In another example, if the sensor system 104 is being implanted in the body post-surgery then the user input may indicate a date/time of surgery, which may indicate that the surgery has already occurred and, therefore, any future lactate concentration measurements received by health monitoring application 106 are going to correspond to the patient's post-surgery lactate concentration levels.

In certain embodiments, instead of mobile computing device 107, another computing system may send an indication to and cause the sensor system 104 to measure the patient's lactate concentration levels. In certain other embodiments, sensor system 104 may itself provide a user interface, such that the user can directly interface with and cause it to begin measuring the patient's lactate concentration levels. In certain embodiments, sensor system 104 may automatically begin to measure the patient's lactate concentration levels upon being implanted in the patient's body.

In certain embodiments, over the pre-sepsis-event time period, sensor system 104 continuously measures the patient's pre-sepsis-event lactate concentration levels and transmits each resulting lactate concentration measurement to health monitoring application 106. The pre-sepsis-event time period may correspond to the entire time the sensor system 104 is operational and implanted in the patient's body prior to the sepsis event or a shorter time period. By the end of this pre-sepsis event time period, therefore, health monitoring application 106 has received a set of pre-sepsis-event lactate concentration measurements.

As described above, this set of pre-sepsis-event lactate concentration measurements can be advantageously used to obtain information about the patient's lactate concentration levels when the patient is not exposed to the risk of sepsis yet or is otherwise in a normal physical state. For example, health monitoring application 106 may use this set of pre-sepsis-event lactate concentration measurements to obtain information including (1) the patient's pre-sepsis-event pattern of lactate levels or changes therein and/or (2) one or more data points including (a) a personalized pre-sepsis-event baseline lactate measurement (“baseline”) for the patient, (b) a standard deviation associated with the patient's pre-sepsis-event surgery lactate concentration measurements, etc. A patient's baseline refers to the average lactate concentration level of the patient when the patient is not experiencing any biological or physiological events that would cause the patient to experience an increase/decrease in lactate levels. The personalized and pre-sepsis-event lactate information, obtained from the set of pre-sepsis-event lactate concentration measurements, can be advantageously used to more accurately identify a risk of sepsis in the patient after the sepsis event, as further described herein.

Once the sepsis event occurs (e.g., the patient undergoes surgery), the same or a different sensor system 104 continuously measures the patient's lactate concentration levels and transmits each resulting lactate concentration measurement to health monitoring application 106. The post-sepsis-event time period may correspond to the entire time the sensor system 104 is operational and implanted in the patient's body after the sepsis event or a shorter time period. During the post-sepsis-event time period, health monitoring application 106, therefore, receives a set of real-time lactate concentration measurements of the patient, which the application 106 uses to monitor the patient for the risk of sepsis.

At block 404, the health monitoring application 106 of system 100 identifies a risk of sepsis in the patient based on the measured lactate concentrations. In certain embodiments, identifying a risk of sepsis may include monitoring the patient for sepsis based on the information described herein. Identifying a risk of sepsis may also include determining a likelihood or possibility of sepsis (e.g., 20%, 90%, very likely, possible, not likely, etc.) or determining whether or not the patient has sepsis in a binary manner (e.g., you have developed sepsis, you do not have sepsis, etc.). Note that although the embodiments herein describe the health monitoring application 106 as the entity or module that performs the operations associated with block 404, in certain embodiments, sensor system 104 may be configured to perform such operations. For example, the sensor electronics (shown in FIG. 7) of sensor system 104 may include a processor able to execute at least some of the instructions/operations described herein with reference to FIG. 4.

In certain embodiments, health monitoring application 106 may utilize a non-personalized approach in identifying sepsis risk in the patient. In such embodiments, health monitoring application 106 may only utilize the patient's post-sepsis-event lactate concentration measurements to determine sepsis risk. In certain other embodiments, as described above, health monitoring application 106 may utilize a personalized approach in identifying sepsis risk in the patient. In such embodiments, health monitoring application 106 may utilize both the patient's post-sepsis-event and pre-sepsis-event lactate concentration measurements to identify sepsis risk. In certain cases, personalizing the identification of sepsis risk is advantageous because, while certain patterns of post-sepsis-event lactate concentration measurements may be indicative of a high risk of sepsis for some patients, in some other patients the same patterns may be relatively normal. Accordingly, analyzing a patient's pre-sepsis-event surgery lactate concentration measurements provides insight into a patient's normal patterns of lactate concentration levels, which can be used to reduce the likelihood of inaccurately identifying a high sepsis risk in the patient post-sepsis-event. Below, a description of the non-personalized approach is first provided followed by a description of the personalized approach.

### Non-Personalized Sepsis Risk Identification

As described above, when utilizing a non-personalized approach to sepsis risk identification, health monitoring application 106 may focus its analysis on the patient's post-sepsis-event (e.g., post surgery) lactate concentration measurements. Generally, because sepsis causes lactate concentration levels to elevate, in certain embodiments, health monitoring application 106 may monitor the patient's post-sepsis-event lactate concentration measurements for an elevated lactate concentration level. In certain embodiments, a threshold-based approach is used to detect an elevated lactate concentration level. For example, health monitoring application 106 may be configured to determine a risk of sepsis in the patient based on whether the patient's post-sepsis-event lactate concentration measurements have reached a defined sepsis threshold. In one example, a lactate concentration level above 2 millimoles (mmol) is considered an important sign of sepsis. As such, in certain embodiments, health monitoring application 106 may identify a risk of sepsis in the patient if health monitoring application 106 receives at least one post-sepsis-event lactate concentration measurement from the sensor system 104 that is equal to or above a sepsis threshold of 2 mmol. Note that, under the non-personalized approach, the defined sepsis threshold is similarly not personalized and may be based on lactate concentration levels generally observed in patients with sepsis. Note that a 2 mmol sepsis threshold is used as an example, and other values (e.g., 1.3 mmol, or 4 mmol) may instead be used.

In certain embodiments, health monitoring application 106 may determine a risk of sepsis based on whether the patient's post-sepsis-event lactate concentration measurements reach or exceed a defined sepsis threshold for at least a minimum duration of time. For example, health monitoring application 106 may identify a risk of sepsis if patient's post-sepsis-event lactate concentration is above 2 mmol for longer than 5 hours. Adding this “minimum duration of time” as a parameter to the sepsis risk analysis may be advantageous as it helps health monitoring application 106 reduce the number of false positives when identifying sepsis risk. To illustrate this with an example, during the post-sepsis-event period, the patient may have an excessively large meal or engage in high intensity exercise, causing the patient's lactate concentration level to exceed 2 mmol. However, in the case of food consumption and exercise, generally, the body stops producing as much lactate or starts clearing the excessive lactate build-up shortly after exercise or food consumption. In other words, when it comes to food consumption and exercise, the body generally experiences an excursion of elevated lactate levels, due to a very high rate of lactate change, followed by a relatively prompt return of the lactate levels to normal ranges.

In contrast, in the case of sepsis, the body experiences a lower but a more sustained rate of lactate change. As a result, the “minimum duration of time” over which the body's lactate concentrations levels are above a certain sepsis threshold is a parameter that can be used to distinguish between non-benign cases (where the patient is experiencing sepsis) and benign cases (food consumption, exercise, or other benign activities). If health monitoring application 106 determines that the post-sepsis-event lactate concentration measurements indicate lactate concentration levels above a threshold for a period longer than the minimum duration of time, then health monitoring application 106 is able to detect sepsis or predict a higher likelihood of sepsis for the patient. In contrast, if the post-sepsis-event lactate concentration measurements indicate lactate concentration levels above the threshold for a period shorter than the minimum duration of time, then health monitoring application 106 may be configured to treat such an event as a non-sepsis related event or simply predict a lower likelihood of sepsis for the patient. Note that another approach for enforcing this “minimum duration of time” is to require at least a certain number of the post-sepsis-event lactate concentration measurements (e.g., counting from the time of the surgery) to be above the sepsis thresholds. In such embodiments, health monitoring application 106 may require that all of such post-sepsis-event lactate concentration measurements be continuous (e.g., without any one of them being below the threshold).

In certain embodiments, health monitoring application 106 may determine a risk of sepsis based on whether the patient's post-sepsis-event lactate concentration measurements indicate a rate of change that is lower than a certain upper threshold. As described above, a high but short-lived rate of change is typically attributable to a non-sepsis event. As such, health monitoring application 106 may determine a high risk of sepsis if patient's post-sepsis-event lactate concentration measurements indicate a rate of change that is, for example, on average less than a defined upper threshold. The defined upper threshold, in certain embodiments, indicates a rate of change that is lower than rates of change that patients, on average, experience after having consumed food or engaged in exercise. In certain embodiments, health monitoring application 106 may also utilize a lower threshold to determine sepsis risk. For example, if the patient's post-sepsis-event lactate concentration measurements indicate a rate of change that is lower than the defined lower threshold, health monitoring application 106 may calculate a low likelihood of sepsis, as the patient's lactate concentrations levels seem to be steady in that example.

In certain embodiments, health monitoring application 106 may not only consider the rate of change but also the duration of time over which the rate of change persists. For example, health monitoring application 106 may determine sepsis risk based on whether the rate of change (e.g., or average rate of change) of the patient's post-sepsis-event lactate concentration measurements has been consistently within the defined range of the lower and upper thresholds, discussed above, for longer than a certain duration. If yes, then health monitoring application 106 calculates a higher risk of sepsis in the patient.

Note that although the non-personalized sepsis risk identification techniques described above involve the use of a patient's post-sepsis-event lactate concentration measurements, in certain embodiments, the same techniques may be used to identify a risk of sepsis for a patient regardless of whether the patient's lactate concentration measurements are post-sepsis-event lactate concentration measurements. For example, these techniques may be used for sepsis risk monitoring for a patient using any plurality of lactate concentration measurements associated with the patient. For example, lactate concentration measurements may be taken for various purposes and used to detect sepsis risk as described herein. These measurements, in various embodiments, may include but are not limited to, pre-sepsis-risk lactate concentration measurements, post-sepsis-risk lactate concentration measurements, continuous lactate concentration measurements, lactate measurements unrelated to sepsis risk, or any combination thereof.

### Personalized Sepsis Risk Identification

When utilizing a personalized approach to sepsis risk identification, health monitoring application 106 may focus its analysis on not only the patient's post-sepsis-risk lactate concentration measurements but also consider the patient's pre-sepsis-risk lactate concentration measurements. As described above, health monitoring application 106 may use the patient's set of pre-sepsis-risk lactate concentration measurements to obtain patient-specific lactate information including (1) the patient's pre-sepsis-risk pattern of lactate levels or changes therein and/or (2) one or more data points including (a) a personalized pre-sepsis-risk baseline lactate measurement (“baseline”) for the patient, (b) a standard deviation associated with the patient's pre-sepsis-risk lactate concentration measurements, etc.

Using this patient-specific lactate information, health monitoring application 106 may better evaluate the risk of sepsis when processing and analyzing the patient's post-sepsis-risk lactate concentration measurements. There are a variety of ways patient-specific lactate information may be used to make more accurate sepsis risk predictions.

In one general example, health monitoring application 106 may determine sepsis risk by comparing the patient's post-sepsis-risk lactate concentration measurements with the patient's pre-sepsis-risk lactate concentration measurements. In such an example, health monitoring application 106 may determine whether a pattern associated with the patient's post-sepsis-risk lactate concentration measurements significantly deviates from a pattern associated with the patient's pre-sepsis-risk lactate concentration measurements. In another example, health monitoring application 106 may determine sepsis risk by determining whether one or more of the patient's post-sepsis-risk lactate concentration measurements exceed the upper bound of a standard deviation associated with the patient's pre-sepsis-risk lactate concentration measurements. If yes, a higher likelihood of sepsis may be calculated, especially if such an event is persistent or lasts for at least a minimum duration of time.

In certain embodiments, certain parameters that may be used for determining sepsis risk, as discussed above, may also be personalized for the patient. For example, the parameters discussed with respect to the non-personalized approach, such as a defined sepsis threshold, the “minimum duration of time,” the lower and upper rate of change thresholds, and the duration of time over which the patient's lactate rate of change persists, etc., may all be personalized. As an example, health monitoring application 106 may be configured to determine a risk of sepsis based on whether the patient's post-sepsis-risk lactate concentration measurements have reached a certain lactate threshold that is calculated based on patient-specific lactate information obtained about the patient pre-sepsis-risk. For instance, the sepsis threshold may be defined or calculated based on the patient's pre-sepsis-risk baseline. In one illustrative example, if the patient's baseline is X, the sepsis threshold may calculated as 2×. In such an example, health monitoring application 106 may, for instance, identify a risk of sepsis in the patient if it receives at least one post-sepsis-risk lactate concentration measurement from the sensor system 104 that is equal to or above 2×.

Note that, as described above, these techniques may be used for sepsis risk monitoring for a patient using any plurality of lactate concentration measurements associated with the patient. For example, lactate concentration measurements may be taken for various purposes and used to detect sepsis risk as described herein. These measurements, in various embodiments, may include but are not limited to, pre-sepsis-risk lactate concentration measurements, post-sepsis-risk lactate concentration measurements, continuous lactate concentration measurements, lactate measurements unrelated to sepsis risk, or any combination thereof.

### Use of Non-Lactate Sepsis Indicators

In certain embodiments, in addition to the use of lactate, health monitoring application 106 may be configured to also use one or more non-lactate sepsis indicators in identifying a risk of sepsis in the patient. Non-lactate sepsis indicators may include one or more of body temperature, heart rate and/or heart rate variability, respiration rate, etc. In certain embodiments, health monitoring application 106 may use one or more of these non-lactate sepsis indicators to verify or confirm the application 106's finding of sepsis risk based on the user's lactate concentration measurements. As an example, if a patient's lactate level is equal to or above 2 mmol and the patient's temperature pattern is atypical, then health monitoring application 106 may determine that the patient has or is developing sepsis. However, if the patient's lactate level is equal to or above 2 mmol, but the patient's temperature pattern is normal, in one example, health monitoring application 106 may refrain from making any prediction about sepsis until additional information is available.

In certain other embodiments, health monitoring application 106 may use a combination of these non-lactate sepsis indicators as well as the patient's lactate concentration measurements to calculate a total likelihood of sepsis. To calculate a likelihood of sepsis, health monitoring application 106 may use a function with weights assigned to each of the lactate and non-lactate indicators. An example of such a function is provided below:

SR=w1(L)+w2(BT)+w3(HR/HRV)+w4(RR)+w5(GM)+

In the function above, SR indicates sepsis risk, L indicates a likelihood of sepsis in the patient based on the patient's lactate measurements, BT indicates a likelihood of sepsis in the patient based on the patient's body temperature information, HR/HRV indicates a likelihood of sepsis in the patient based on the patient's heart rate or heart rate variability information, RR indicates a likelihood of sepsis in the patient based on the patient's respiratory rate information, and GM indicates a likelihood of sepsis in the patient based on the patient's glucose measurement information. The weights also correspond to the correlations between the sepsis indicators and the likelihood of sepsis. For example, as lactate concentration levels of a patient may be the best indicator or predictor of sepsis risk, w1 may be larger than the other weights in the example function above. In one example, if the sum of all the weighted likelihoods exceeds a threshold then health monitoring application 106 determines that the patient has sepsis. Note that the function above is merely exemplary and is shown to illustrate that a combination of lactate and non-lactate sepsis indicators may be used to more accurately detect or predict the risk of sepsis in a patient. A brief description of each of the non-lactate sepsis indicators is provided below.

### Body Temperature

An atypical body temperature pattern is another sign of sepsis. In certain embodiments, an atypical body temperature pattern may indicate a drastic and/or sudden (e.g., high rate of change) in temperature or a pattern thereof over a certain time period (e.g., past 24 hours). In certain embodiments, an atypical body temperature pattern may indicate a body temperature of above about 101 degrees F. or below about 97 degrees F. In certain embodiments, body temperature measurements may be manually inputted into health monitoring application 106. In certain other embodiments, in addition to a lactate sensor, a body temperature sensor may be provided as part of the sepsis monitoring system 100. The body temperature sensor may be configured to continuously measure the patient's body temperature and transmit the body temperature measurements in real-time to health monitoring application 106.

The body temperature sensor can be part of the lactate sensor or the lactate sensor electronics of sensor system 104. In certain embodiments, if the body temperature sensor is provided as part of sensor system 104, sensor system 104 may be implanted in an area of the body where temperature measurements can be correlated to the core body temperature. A “measurement” of body temperature need not be made directly as a result of the temperature sensor contacting internal organs or body cavities. The raw data of skin temperatures and the like can be calibrated to become a sufficiently accurate body temperature measurement based on relationships between body core temperature and the temperature directly measured by a temperature sensor associated with the lactate sensor or sensor electronics.

It may be noted that it is currently common practice to take measurements of the ambient temperature in vivo and/or ex vivo on or near an implanted blood analyte sensor. In these conventional applications, this data is used to compensate the acquired sensor signal for temperature changes because the sensitivity of the sensor can be temperature dependent. As such, these conventional temperature measurements are not body temperature measurements. There is no need for temperature data acquired and used for sensor signal compensation to be the same as or even related to the body temperature of the patient. The requirement is that the temperature data be a measurement of the sensor environment, whatever that happens to be. For the present sepsis risk monitoring application, additional measures will be taken to relate the temperature measurements to the actual body temperature of the patient. As noted above, this may be done by implanting the sensor in an appropriate location, or by correcting the actual measurement with a known relationship between measured temperature and patient body temperature or a combination of both for example. These steps are not performed and are not needed for conventional temperature compensation.

### Heart Rate

Heart rate can advantageously be used in identifying sepsis risk. For example, an abnormally high heart rate may be an indication of sepsis. In another example, a drop in heart rate variability of more than a defined threshold may be used as an indication of sepsis. For example, a 25% (or higher) drop in heart rate variability may be an indication of sepsis. In certain embodiments, a low and persisting heart rate variability may be an even stronger indication of sepsis. For example, health monitoring application 106 may assign a higher likelihood of sepsis to a patient who experiences a low heart rate variability for at least a defined duration of time (e.g., at least X number of hours) than if the patient experienced the same heart rate variability over a much shorter period of time.

In certain embodiments, a heart rate sensor may be provided as part of the sepsis monitoring system 100. For example, a heart rate sensor may be worn on the wrist or chest and communicate wirelessly with sensor system 104. In certain other embodiments, a heart rate sensor (e.g., photoplethysmogram (PPG) sensor) may be provided as part of the sensor system 104 (e.g., embedded in the lactate sensor). For example, the heart rate sensor may be part of the lactate sensor or the sensor electronics of sensor system 104.

### Respiration Rate

Generally, an abnormally high respiration rate may be an indication of sepsis. In certain embodiments, a respiration rate sensor may be provided as part of the sepsis monitoring system 100. For example, a respiration rate sensor may be worn on the chest and communicate wirelessly with sensor system 104. In certain other embodiments, a respiration rate sensor may be provided as part of the sensor system 104. For example, the respiration rate sensor (e.g., photoplethysmogram (PPG) sensor) may be part of the lactate sensor (e.g., embedded in the lactate sensor) or the sensor electronics of sensor system 104.

### Distinguishing Sepsis from Other Events

As discussed, in certain cases, non-sepsis events, such as food consumption, exercise, etc., may also cause a patient's lactate levels to elevate. As described with respect to the personalized and non-personalized techniques for sepsis risk identification, health monitoring application 106 may be configured with algorithms to distinguish between lactate elevation patterns that correspond to sepsis versus exercise or food consumption. More specifically, in certain embodiments, the algorithms used with respect to personalized and non-personalized techniques described above, may distinguish between sepsis and food/exercise based on metrics such as rate of change of lactate, the duration over which the rate of change exceeds a certain sepsis threshold, etc. However, to more accurately calculate sepsis risk and/or to confirm any determinations made based on such algorithms, in certain embodiments, health monitoring application 106 may use one or more additional parameters. Examples of such parameters are heart rate, glucose measurements, accelerometer, user input, etc. For example, a high heart rate measurement (although not abnormally high) may indicate that the patient has or is engaged in exercise and, therefore, the patient's elevated lactate levels may not be due to sepsis. In a similar example, output from an accelerometer may also be used in combination with the patient's heart rate to determine whether the patient has or is engaged in exercise.

In certain embodiments, the lactate sensor may be compressed into the patient's body, causing the localized lactate concentration levels to raise. As such, one or more compression detection techniques may be utilized to determine if the patient's elevated lactate levels are due to sepsis or compression. For example, one or more sensors may be used to determine whether the patient is asleep. For example, in one embodiment, a patient who is asleep is more likely to be in a position where the lactate sensor would be compressed into his/her body. One example sensor is an orientation sensor that may be used to detect whether the patient's orientation is horizontal. Other sensors include respiratory, heartbeat, movement, etc., sensors that can indicate whether the patient is sleeping. In certain embodiments, a glucose sensor may also provide glucose measurements that can be indicative of compression. This is because, in the event of compression, both lactate and glucose levels increase. Therefore, an increase in both lactate and glucose levels may be an indication of compression.

In certain embodiments, glucose measurements may be used to determine whether the patient just engaged in exercise or consumed food. For example, after a meal, the patient may experience not only an increase in lactate levels but also an increase in glucose levels. As such, in situations where health monitoring application 106 receives indications of both elevated lactate and glucose levels, the application 106 may, in one example, calculate a lower likelihood of sepsis than if only lactate levels had elevated.

Health monitoring application 106 may similarly use user input to determine if a patient's elevated lactate levels are likely due to sepsis or other events, such as exercise or food consumption. For example, if the user of the health monitoring application 106 provides user input indicating that the patient just engaged in exercise or consumed food, then health monitoring application 106 may calculate a lower likelihood of sepsis. In certain embodiments, user input may be used as confirmation for what health monitoring application 106 has decided using one of more of the other parameters above. In one non-limiting example, if health monitoring application 106 observes that the patient's lactate as well as glucose levels are elevating but that the patient's lactate elevation pattern does not perfectly correspond to lactate patterns associated with sepsis, the health monitoring application 106 may determine that it is highly likely that the patient just consumed food. To confirm this determination, health monitoring application 106 may query the user as to whether the patient in fact just consumed food. If the user responds negatively, then health monitoring application 106 may recalculate (e.g., increase) the risk of sepsis. If the user responds positively, then application 106's prior sepsis risk calculations may remain unchanged or application 106 may even reduce the risk of sepsis.

The above example is merely to illustrate how a combination of two parameters (i.e., glucose measurements and user input) are used for sepsis risk identification. However, there are a variety other ways a combination of two or more of the parameters above may be used by health monitoring application 106 to distinguish between sepsis and other benign events.

Note that although in certain embodiments described above user input is used to determine or confirm whether the patient's elevated lactate levels are due to sepsis or other events, in certain embodiments user input is used as an indication of how the user is feeling in real-time. For example, if health monitoring application 106 observes a pattern of elevated lactate levels, it may query the user to determine how the user is feeling. If the user's input indicates that the user is physically not feeling well, then such an indication may be used to increase the likelihood that the patient has sepsis or vice versa.

### Sepsis Risk Identification Algorithms

There are a variety of algorithms and functions (some of which were described above) that may be used to determine sepsis risk based on the lactate concentration measurements as well as non-lactate parameters. The non-lactate parameters may include the non-lactate sepsis indicators described above (e.g., body temperature, heart rate and/or heart rate variability, respiration rate, etc.) as well as glucose measurements, accelerometer information, user input, etc. In certain embodiments, as described above, each of the non-lactate parameters may be assigned corresponding weights and used in an algorithm or a function, such as the SR function described above, to calculate a risk of sepsis. In one example, as described above, if the sum of all the weighted likelihoods exceeds a threshold then health monitoring application 106 determines that the patient has sepsis. In certain embodiments, one or more decision trees may instead or in addition be used.

Referring back to flow chart 400, once a risk of sepsis is identified, at block 406, system 100 provides an indication to a user based on the identified risk of sepsis. Providing an indication to a user of application 106 may include providing an audible and/or visual alert, notification, etc. The audible and/or visual alert or notification may differ in characteristics (e.g., shape, format, color, font, sound level, etc.), depending on how likely it is that the patient is has developed sepsis. In addition, the frequency with which the indication is provided to the user may vary based on the likelihood that the patient has developed sepsis. The higher the likelihood, the higher the frequency. Note that although the embodiments herein describe the health monitoring application 106 as the entity or module that performs the operations associated with block 406, in certain embodiments, sensor system 104 may be configured to perform such operations.

In certain embodiments, providing an indication to a user of health monitoring application 106 includes providing a likelihood of the patient developing sepsis. In one example, health monitoring application 106 may provide one of the following outputs to the user: (1) it is very likely that you are have developed sepsis or in the early stages of developing sepsis, (2) it is likely that you have developed sepsis sepsis or in the early stages of developing sepsis, (3) it is unlikely that you have developed sepsis or in the early stages of developing sepsis. Each of these outputs may be provided to the user using a user interface feature with a shape, format, color, or font that is different from the other user interface features associated with other outputs. For example, if output (1) is selected, the shape, format, color, or font of the user interface used to provide output (1) to the user may be chosen specifically to put the user on high alert. As an example, a font used for the user interface feature associated with output (1) may be bigger than a font used for the user interface feature associated with output (3). Instead of user interface features, these outputs may also be provided to the user audibly with different sound levels depending on which output is being provided.

In certain embodiments, providing an indication to a user of health monitoring application 106 includes providing a percentage risk of the patient having developed sepsis. In such an example, health monitoring application 106 may output an indication to the user that is indicative of the percentage (e.g., it is 90% likely that you are have developed sepsis).

Providing an indication to the user of health monitoring application 106 may also include a binary output. For example, health monitoring application 106 may indicate one of the following to the patient: (1) you have developed sepsis or (2) you have not developed sepsis. In certain embodiments, in the event that there is a high risk of sepsis in the patient, health monitoring application 106 may further alert the clinician or the clinic to reach out to the patient, make an appointment for a visit, send an ambulance, etc.

Providing an indication to the user may include the use of a user interface provided by sensor system 104. Examples of the types of user interface that may be provided by sensor system 104 are described in further detail below.

In certain embodiments, it is advantageous to optimize the lactate sensor construction for the specific use of post-sepsis-event sepsis risk monitoring. FIG. 5A shows one exemplary embodiment of the physical structure of lactate sensor 538. In this embodiment, a radial window 503 is formed through an insulating layer 505 to expose an electroactive working electrode of conductor material 504. Although FIG. 5A shows a coaxial design, any form factor or shape such as a planar sheet may alternatively be used. A variety of lactate sensor designs are described in Rathee et al. “Biosensors based on electrochemical lactate detection: A comprehensive review,” Biochemistry and Biophysics Reports 5 (2016) pages 35-54, and also Rasaei et al. “Lactate Biosensors: current status and outlook” in Analytical and Bioanalytical Chemistry, September 2013, both of which are incorporated herein by reference in their entireties.

FIG. 5B is a cross-sectional view of the electroactive section of the example sensor of FIG. 5A showing the exposed electroactive surface of the working electrode surrounded by a sensing membrane in one embodiment. Such sensing membranes are present in a variety of lactate sensor designs. As shown in FIG. 5B, a sensing membrane may be deposited over at least a portion of the electroactive surfaces of the sensor (working electrode and optionally reference electrode) and provides protection of the exposed electrode surface from the biological environment, diffusion resistance of the analyte, a catalyst for enabling an enzymatic reaction, limitation or blocking of interferants, and/or hydrophilicity at the electrochemically reactive surfaces of the sensor interface.

Thus, the sensing membrane may include a plurality of domains, for example, an electrode domain 507, an interference domain 508, an enzyme domain 509 (for example, including lactate oxidase), and a resistance domain 500, and can include a high oxygen solubility domain, and/or a bioprotective domain (not shown). The membrane system can be deposited on the exposed electroactive surfaces using known thin film techniques (for example, spraying, electro-depositing, dipping, or the like). In one embodiment, one or more domains are deposited by dipping the sensor into a solution and drawing out the sensor at a speed that provides the appropriate domain thickness. However, the sensing membrane can be disposed over (or deposited on) the electroactive surfaces using any known method as will be appreciated by one skilled in the art.

The sensing membrane generally includes an enzyme domain 509 disposed more distally situated from the electroactive surfaces than the interference domain 508 or electrode domain 507. In some embodiments, the enzyme domain is directly deposited onto the electroactive surfaces. In the preferred embodiments, the enzyme domain 509 provides an enzyme such as lactose oxidase to catalyze the reaction of the analyte and its co-reactant.

The sensing membrane can also include a resistance domain 500 disposed more distal from the electroactive surfaces than the enzyme domain 509 because there exists a molar excess of lactate relative to the amount of oxygen in blood. However, an enzyme-based sensor employing oxygen as co-reactant is preferably supplied with oxygen in non-rate-limiting excess for the sensor to respond accurately to changes in analyte concentration rather than having the reaction unable to utilize the analyte present due to a lack of the oxygen co-reactant. This has been found to be an issue with glucose concentration monitors and is the reason why the resistance domain is included. Specifically, when a glucose-monitoring reaction is oxygen limited, linearity is not achieved above minimal concentrations of glucose. Without a semipermeable membrane situated over the enzyme domain to control the flux of glucose and oxygen, a linear response to glucose levels can be obtained only for glucose concentrations of up to about 2 or 3 mM. However, in a clinical setting, a linear response to glucose levels is desirable up to at least about 20 mM. To allow accurate determination of higher glucose levels, the resistance domain in the glucose monitoring context can be 200 times more permeable to oxygen than glucose. This allows an oxygen concentration high enough to make the glucose concentration the determining factor in the rate of the detected electrochemical reaction.

In some embodiments, for the lactate sensors described herein, the resistance domain can be thinner, and have a smaller difference in analyte vs. oxygen permeability, such as 50:1, or 10:1 oxygen to lactate permeability. In some embodiments, this makes the lactate sensor more sensitive to low lactate levels such as 0.5 mM or lower up to 3 or 4 mM. The resistance domain may be configured such that lactate is the rate limiting reactant at 3 mM lactate or lower, thus allowing accurate threshold detection at around 2 mM. The resistance domain may further be configured to allow oxygen to be the rate limiting reactant at lactate concentrations greater than 10 mM. These ranges may be narrowed further in some embodiments, for example the resistance domain may be configured such that lactate is the rate limiting reactant at 4 mM lactate or lower, and such that oxygen is the rate limiting reactant at lactate concentrations greater than 6 mM. In this way, the sensor itself can be optimized for early sepsis detection. It will also be appreciated that in addition to lactate, other analyte sensors can be combined with the lactate sensor described herein, such as sensors suitable for ketones, ethanol, glycerol, glucose, hormones, viruses, or any other biological component of interest.

FIGS. 6A, 6B, and 6C illustrate an exemplary implementation of a sensor system 104 implemented as a wearable device such as an on-skin sensor assembly 600. As shown in FIGS. 6A and 6B, on-skin sensor assembly comprises a housing 628. An adhesive patch 626 can couple the housing 628 to the skin of the host. The adhesive 626 can be a pressure sensitive adhesive (e.g., acrylic, rubber based, or other suitable type) bonded to a carrier substrate (e.g., spun lace polyester, polyurethane film, or other suitable type) for skin attachment. The housing 628 may include a through-hole 680 that cooperates with a sensor inserter device (not shown) that is used for implanting the sensor 538 under the skin of a subject.

The wearable sensor assembly 600 includes sensor electronics 635 operable to measure and/or analyze lactate concentration indicators sensed by lactate sensor 538. As shown in FIG. 6C, in this implementation the sensor 538 extends from its distal end up into the through-hole 680 and is routed to a sensor electronics 635, typically mounted on a printed circuit board 635 inside the enclosure 628. The sensor electrodes are connected to the sensor electronics 635. These kinds of analyte monitors are currently used in commercially available glucose monitoring systems used by diabetics, and the design principles used there can be used for an lactate monitor as well.

The housing 628 of the sensor assembly 600 can include a user interface for delivering messages to the patient regarding sepsis status. Because the lactate sensors described herein may, in some examples, not be a monitor that a patient will wear regularly as is the case with glucose monitors, in such examples, they may not need to include many of the features present in other monitor types such as regular wireless transmission of analyte concentration data. Accordingly, a simple user interface to just deliver warnings can be implemented. In some embodiments, the user interface could be a single light-emitting diode (LED) that is illuminated when the sensor electronics determines sepsis risk is present. Two LEDs or a two-color LED could be green when the monitor is operational and detects low risk, and red when a sepsis risk is detected and a warning is issued. The monitor may be configured to revert back to a green or low risk condition if measurements return to values appropriate for that output. To provide additional flexibility in delivering messages to patients such as error messages, time remaining to wear the device, etc., a simple dot matrix character display could be used (for example less than 200 pixels a side or a configurable 20 character LCD) that would still be inexpensive and power efficient.

In some embodiments, simple patient feedback could be received that would be valuable in accurately assessing sepsis risk. The monitor may have a button on the housing that the user can press if they feel ill. How the patient feels is another important aspect of sepsis diagnosis, and this input can be used to further refine the warning issuance algorithm. If the monitor has a simple character display, it could ask the user to press one or more buttons on the device to indicate how they are feeling. A combination of lactate concentration, body temperature, subjective patient input concerning whether they feel healthy or not, as well as the other parameters (e.g., non-lactate parameters) constitutes a powerful combination of sepsis diagnosis factors.

The monitors described herein are not primarily intended to deliver a diagnosis of sepsis that medical personnel receive or to provide clinical decision support during in- hospital treatment of sepsis. As noted above, it would be expected that conventional lactate monitoring and sepsis diagnosis and treatment according to long-standing practice would continue at the health care facility. Instead, these lactate monitors are primarily intended for telling patients that they should seriously consider having their condition reviewed by professionals.

FIG. 7 is a block diagram that illustrates example sensor electronics 732, also referred to as sensor electronics and/or an electronics module, associated with the sensor system 104 of FIG. 1. In this embodiment, a potentiostat 734 is shown, which is operably connected to an electrode system (such as described above) and provides a voltage to the electrodes, which biases the sensor to enable measurement of a current signal indicative of the analyte concentration in the patient (also referred to as the analog portion). In some embodiments, the potentiostat includes a resistor (not shown) that translates the current into voltage. In some alternative embodiments, a current to frequency converter is provided that is configured to continuously integrate the measured current, for example, using a charge counting device. An A/D converter 136 digitizes the analog signal into a digital signal for processing. Accordingly, the resulting raw data stream is directly related to the current measured by the potentiostat 734.

A processor module or processor 738 includes a central control unit that controls the processing for the sensor electronics 732. In some embodiments, the processor 738 includes a microprocessor, ASIC, DSP, microcontroller, FPGA, or the like. The processor 738 typically provides semi- permanent storage of data, for example, storing data such as sensor identifier (ID) and programming to process data streams (for example, programming for data smoothing and/or replacement of signal artifacts. The processor 738 additionally can be used for the system's cache memory, for example for temporarily storing recent sensor data. In some embodiments, the processor 738 comprises memory storage components such as ROM, RAM, dynamic RAM, static-RAM, non-static RAM, EEPROM, rewritable ROMs, flash memory, or the like. In some embodiments, the processor 738 stores instructions (e.g., health monitoring application), that when executed, cause sensor electronics 732 to perform one or more of the operations (e.g., blocks) associated with the method illustrated in FIG. 4. For example, the processor 738 may store instructions to identify a risk of sepsis (as described in relation to block 404) and provide an indication to the user based on the identified risk of sepsis (e.g., as described in relation to block 406). In certain embodiments, sensor electronics 732 may provide the indication to the user using a display, monitor, and/or user interface described with reference to FIGS. 6A-6B above. The display, monitor, and/or user interface may be provided as part of or be coupled to sensor electronics 732.

In some embodiments, the processor 738 is configured to smooth the raw data stream from the A/D converter. Generally, digital filters are programmed to filter data sampled at a predetermined time intervals (also referred to as a sample rate). In some embodiments, the potentiostat is configured to measure the analyte at discrete time intervals, wherein these time intervals determine the sample rate of the digital filter. In some embodiments, the potentiostat is configured to continuously measure the analyte, for example, using a current-to-frequency converter as described above. The processor 738 can be programmed to request a digital value from the A/D converter at a predetermined time interval, also referred to as the acquisition time. In certain embodiments, the values obtained by the processor 738 may be advantageously averaged over the acquisition time due the continuity of the current measurement. Accordingly, the acquisition time determines the sample rate of the digital filter. In some embodiments, the processor 738 is configured with a programmable acquisition time.

A power source, such as a battery 744, is operably connected to the sensor electronics 732 and provides the power for at least one of the lactate sensor and the sensor electronics, typically both. In certain embodiments, the battery is a lithium manganese dioxide battery; however, any appropriately sized and powered battery can be used (for example, AAA, nickel-cadmium, zinc carbon, alkaline, lithium, nickel-metal hydride, lithium-ion, Zinc- air, zinc-mercury oxide, silver-zinc, and/or hermetically-sealed).

Temperature probe 740 is shown, wherein the temperature probe 740 is located ex vivo in or on the sensor electronics 732 or in vivo on the lactate sensor itself, or any other suitable location for measuring the patient's body temperature. As described above, this body temperature measurement can be integrated with the lactate concentration measurement so that the two together can be used in an algorithm defining when a warning will be delivered to a patient. As described above, sensor system 104 may also include a heart rate sensor (not shown), a respiration sensor (not shown), an accelerometer (not shown), a continuous glucose monitoring sensor (not shown), etc., that are able to provide corresponding measurements that may be used to more accurately identify sepsis risk.

In some implementations, an RF module 748 is operably connected to the processor 738 and transmits the sensor data from the sensor to a receiver such as mobile computing device 107 via antenna 752. In some embodiments, a second quartz crystal 754 provides the time base for the RF carrier frequency used for data transmissions from the RF transceiver. In some alternative embodiments, however, other mechanisms, such as optical, infrared radiation (IR), ultrasonic, or the like, can be used to transmit and/or receive data. In general, the RF module 748 includes a radio and an antenna, wherein the antenna is configured for radiating or receiving an RF transmission. In some embodiments, the radio and antenna are located within the electronics unit. In some embodiments, the sensor electronics 732 is coupled to an RFID or similar chip that can be used for data, status or other communications.

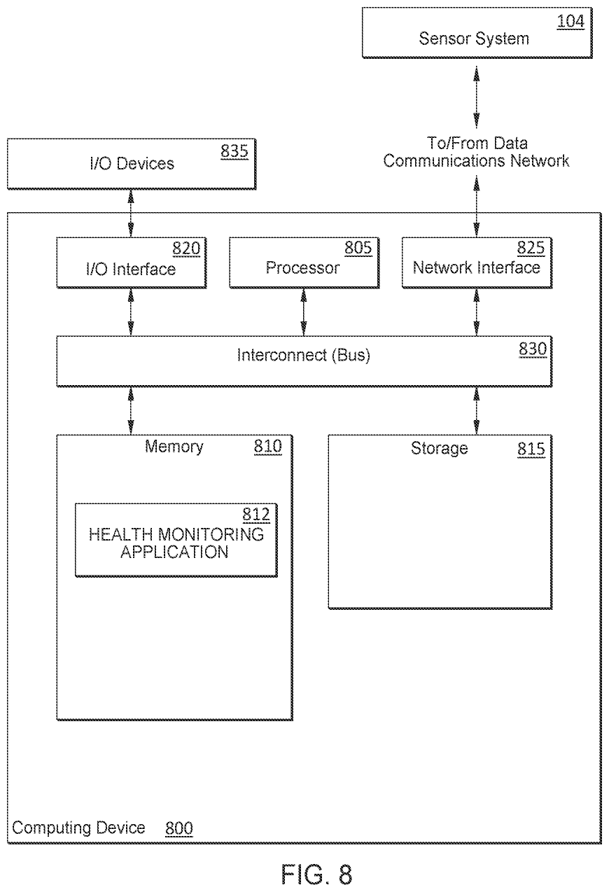

FIG. 8 is a block diagram depicting a computing device 800 (e.g., mobile computing device 107) configured to perform health monitoring, according to certain embodiments disclosed herein. Although depicted as a single physical device, in embodiments, computing device 800 may be implemented using virtual device(s), and/or across a number of devices, such as in a cloud environment. As illustrated, computing device 800 includes a processor 805, memory 810, storage 815, a network interface 825, and one or more I/O interfaces 820. In the illustrated embodiment, processor 805 retrieves and executes programming instructions stored in memory 810, as well as stores and retrieves application data residing in storage 815. Processor 805 is generally representative of a single CPU and/or GPU, multiple CPUs and/or GPUs, a single CPU and/or GPU having multiple processing cores, and the like. Memory 810 is generally included to be representative of a random access memory. In the illustrated embodiment, memory 610 stores health monitoring application 106. Storage 815 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, input and output (I/O) devices 835 (such as keyboards, monitors, speakers, etc.) can be connected via the I/O interface(s) 820. Further, via network interface 825, computing device 800 can be communicatively coupled with one or more other devices and components, such sensor system 104. In certain embodiments, computing device 800 may be configured with hardware/software (e.g., RF transceiver) necessary to communicate with sensor system 104 wirelessly, such as through Bluetooth, near field communications (NFC), or other wireless protocols. In certain embodiments, computing device 800 is communicatively coupled with other devices via a network, which may include the Internet, local network(s), and the like. The network may include wired connections, wireless connections, or a combination of wired and wireless connections. As illustrated, processor 805, memory 810, storage 815, network interface(s) 825, and I/O interface(s) 820 are communicatively coupled by one or more interconnects 830. In certain embodiments, computing device 800 is representative of mobile device 107 associated with the user. In certain embodiments, as discussed above, the mobile device 107 can include the user's laptop, computer, smartphone, and the like.

Accordingly, certain embodiments described herein improve the technical field of sepsis risk monitoring. As discussed, the sensor system described herein enables sepsis monitoring to occur even when the patient is not at a healthcare facility. Without the use of a continuous lactate sensor, sepsis risk may be increased and more difficult to detect when the patient is not at a healthcare facility and not being actively monitored by a clinician.

Further, using the wearable sensor system described herein removes the delay associated with obtaining lactate concentration information from blood draws (e.g., finger sticks), therefore, allowing for sepsis risk monitoring to be performed based on real-time lactate concentration levels of the patient. Also, because the sensor system described herein continuously measures the patient's lactate concentration levels (e.g., much more frequently than periodic blood draws), trends and patterns can be established that may not only be used for early and more accurate detection of sepsis but also to determine whether a patient is responding to treatment in real-time. Earlier and more accurate detection of sepsis allows for earlier and more effective intervention.

In addition, the use of the sensor system described herein allows for identifying sepsis risk at higher accuracy rates by utilizing personalized sepsis monitoring techniques involving analysis around the patient's pre-sepsis-event lactate concentration levels. Further, the algorithms and methods described herein improve the functionality of a health monitoring system, which may include a sensor system and/ a computing device, for identifying sepsis risk.

### Athletic Performance Monitoring and Evaluation

In addition to sepsis monitoring, health monitoring application 106 may be configured to perform athletic performance monitoring based on lactate concentration measurements of a user.

As described above, during strenuous physical activity, muscles utilize multiple metabolic energy systems to sustain physical activity. In these cases, the muscle tissue will utilize aerobic and anaerobic metabolic pathways that result in the net accumulation of lactate in the body. Athletic performance is correlated to the amount of work the muscles can do before the accumulation of lactate occurs. The greater the work that can be performed prior to the accumulation of lactate, the better the athlete is able to perform and the higher their metabolic fitness.

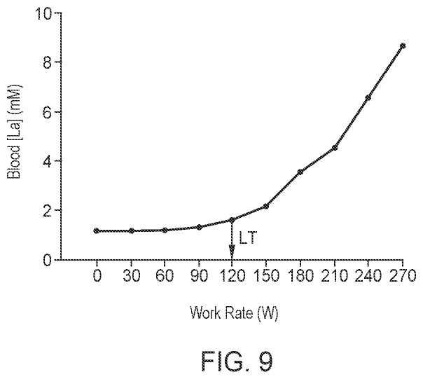

FIG. 9 shows a typical determination of “lactate threshold” for an athlete. To determine lactate threshold, an athlete will get on a treadmill or exercise bicycle and be subjected to incrementally increased work load. Blood is periodically drawn during the test and the lactate concentration is measured. There will typically be a work load where lactate concentrations start to increase at a high rate, e.g., an inflection point labeled LT in FIG. 9. Successful training regimens increase this threshold, and the threshold forms a data point in a fitness evaluation.

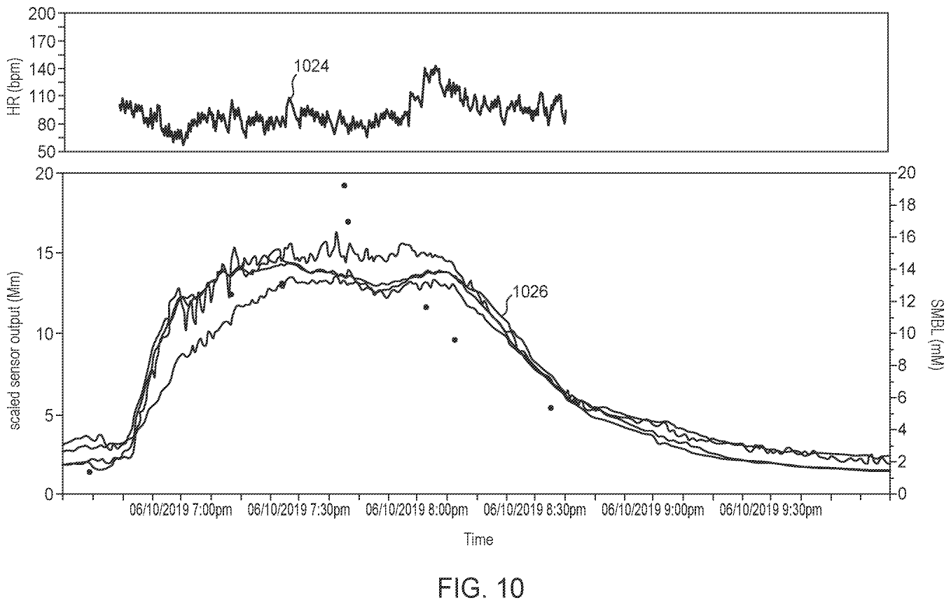

FIG. 10 shows lactate levels 1026 and heart rate 1024 measured for a subject over about a two-hour resistance training workout. As can be seen, for these types of workouts that are not focused on the cardiovascular and respiratory systems, heart rate is a poor measure of intensity of workload. It can also be seen that even though resistance training tends to target localized muscle groups, there is still a systemic lactate increase that can be measured. For this workout, the subject wore four different transcutaneous lactate sensors having two different lactate oxidase sources and being placed on two different body locations, abdomen and arm. The individual dots are individual blood draws applied to lactate test strips during the workout.

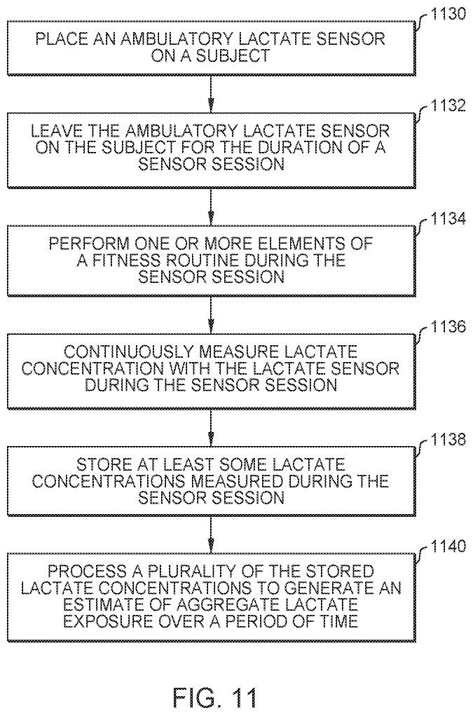

FIG. 11 is one example embodiment of using sensor system 104 as a fitness training aid. In this embodiment, the sensor system 104, which may be transcutaneous or non-invasive, is applied to a subject. The sensor system 104 is applied for a duration defining a sensor session. Elements of a fitness routine are performed during the sensor session as lactate concentrations are recorded. In contrast with conventional lactate threshold testing, a sensor session will in some embodiments span multiple elements of a fitness routine, often over several days such as three days, ten days, or more. As shown at block 1140, lactate concentration recorded over the sensor session can be used to generate an estimate of aggregate lactate load over part of or the whole sensor session. For example, if lactate levels are measured every minute during a sensor session, the aggregate lactate load could be defined as the sum of all the individual lactate measurements divided by the number of measurements made, defining something that may be seen as “lactate-minutes” of elevated lactate (e.g., development of high concentration of lactate in the body) over the sensor session. Refinements of an algorithm such as this may include setting lactate measurements below a threshold such as 2 or 5 millimoles per liter (mM) to zero for purposes of the computation.

This method allows an entire extended fitness routine to be quantified in terms of its intensity for the subject. With this information, fitness routines can be modified to target levels or ranges of intensity defined by overall extended lactate load.

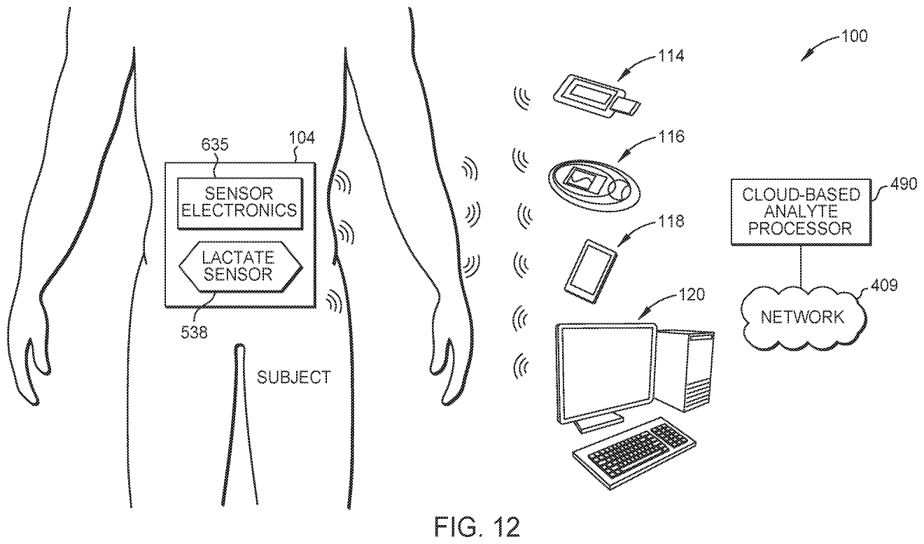

FIG. 12 shows an exemplary sensor system 104 where a lactate sensor 538 communicates with sensor electronics 112. The sensor electronics can process data on board or may send it to other devices 114, 116, 118, and 120 for processing.

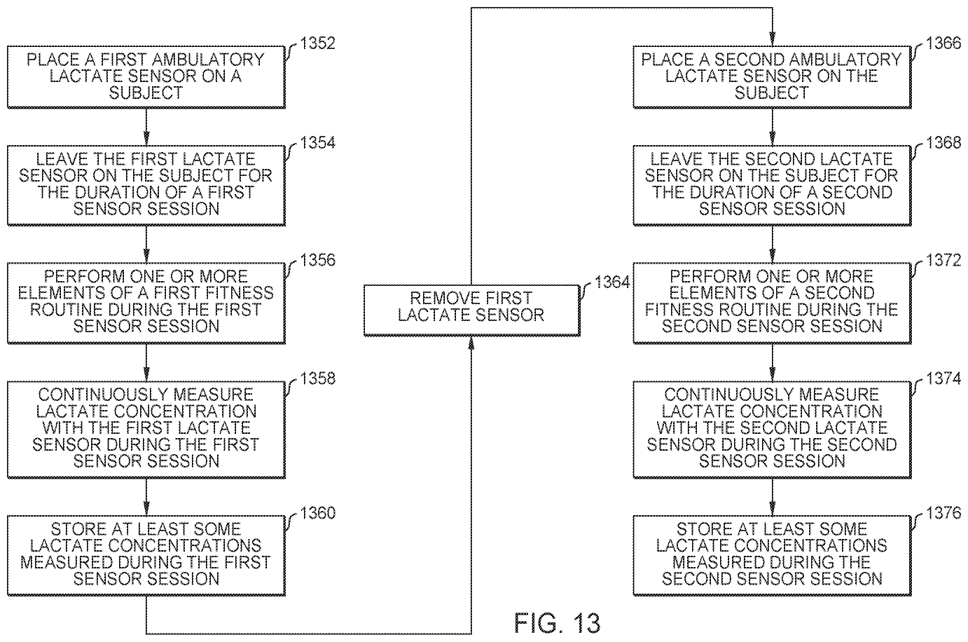

FIG. 13 is a second embodiment of a method of using lactate sensing as a fitness training aid. In this embodiment, two sensor sessions are used with potentially different fitness routines. Lactate loads for the different sessions can be compared and fitness routines may be modified according to the result.

### General Interpretive Principles for the Present Disclosure

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure.

For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein

The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub- combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

### Example Embodiments

Example Embodiment 1 includes a method of activity monitoring comprising: implanting a transcutaneous lactate sensor; leaving the transcutaneous lactate sensor implanted for the duration of a sensor session; performing one or more elements of a fitness routine during the sensor session; continuously measuring lactate concentration with the transcutaneous lactate sensor during the sensor session; storing at least some lactate concentrations measured by the transcutaneous lactate sensor during the sensor session.

Example Embodiment 2 includes the method of Example Embodiment 1, wherein the sensor session lasts at least twelve hours.

Example Embodiment 3 includes the method of Example Embodiments 2 and 3, wherein a plurality of elements of the fitness routine are performed during the sensor session.

Example Embodiment 4 includes the method of Example Embodiment 3, wherein at least two of the one or more elements of the fitness routine are separated by at least six hours.

Example Embodiment 5, wherein the sensor session lasts at least ten days.

Example Embodiment 6, wherein the lactate sensor is operably connected to sensor electronics, wherein the sensor electronics comprises memory, and wherein the storing comprises storing in the memory of the sensor electronics.

Example Embodiment 7 includes the method of Example Embodiment 6, comprising transmitting stored lactate concentrations to a separate device.

Example Embodiment 8 includes the method of Example Embodiment 7, wherein the separate device comprises a smartphone.

Example Embodiment 9, comprising processing a plurality of lactate concentrations measured by the lactate sensor to generate an estimate of aggregate lactate over a period of time.

Example Embodiment 10 includes the method of Example Embodiment 9, wherein the period of time is selected by a user of the lactate sensor.

Example Embodiment 11, wherein the period of time is the duration of the sensor session.

Example Embodiment 12, comprising processing a plurality of lactate concentrations measured by the lactate sensor to generate an estimate of a peak lactate over a period of time.

Example Embodiment 13 including a method of activity monitoring comprising: placing a first lactate sensor on a subject; leaving the lactate sensor implanted for the duration of a first sensor session; performing one or more elements of a first fitness routine during the first sensor session; continuously measuring lactate concentration with the lactate sensor during the first sensor session; storing at least some first lactate concentrations measured by the lactate sensor during the first sensor session; removing the first lactate sensor from the subject; placing a second lactate sensor on the subj ect after removing the first ambulatory lactate sensor; leaving the second lactate sensor implanted for the duration of a second sensor session; performing one or more elements of a second fitness routine during the second sensor session; continuously measuring lactate concentration with the second lactate sensor during the second sensor session; storing at least some second lactate concentrations measured by the lactate sensor during the second sensor session.

Example Embodiment 14 including the method of Example Embodiment 13, wherein the both the first and second sensor sessions last at least twelve hours.

Example Embodiment 15, wherein a plurality of elements of the first fitness routine are performed during the first sensor session and wherein a plurality of the elements of the second fitness routine are performed during the second sensor session.

Example Embodiment 16, wherein the second fitness routine is different from the first fitness routine.

Example Embodiment 17, wherein at least one element of the first fitness routine is performed as part of the second fitness routine.

Example Embodiment 18, wherein differences between the first fitness routine and the second fitness routine are based at least in part on the stored first lactate concentrations measured by the transcutaneous lactate sensor at least during the performing of the first fitness routine.

Example Embodiment 19, wherein the average lactate of the second sensor session is greater than the average lactate of the first sensor session.

Example Embodiment 20, wherein the difference in average lactate of the second sensor session is due at least in part by the differences between the first fitness routine and the second fitness routine that are based at least in part on the stored first lactate concentrations measured by the transcutaneous lactate sensor at least during the performing of the first fitness routine.

Example Embodiment 21, including an activity monitoring system comprising: an ambulatory lactate sensor; sensor electronics operably connected to the ambulatory lactate sensor; a memory operably connected to the sensor electronics for storing measured lactate concentrations; a processor configured to generate an estimate of aggregate lactate over a period of time based at least in part on stored measured lactate concentrations.

Example Embodiment 22, wherein the lactate sensor is a transcutaneous sensor.

Example Embodiment 23, wherein the lactate sensor is a non-invasive sensor.

Example Embodiment 24, wherein the memory is part of the sensor electronics.

Example Embodiment 25, wherein the memory is part of a separate device.

Example Embodiment 26, wherein the processor is part of the sensor electronics.

Example Embodiment 27, wherein the processor is part of a separate device.

Example Embodiment 28, wherein the separate device is a smartphone.

Example Embodiment 29, including a method of activity monitoring comprising: placing a lactate sensor on a subject; leaving the lactate sensor on the subject for the duration of a sensor session; performing a plurality of elements of a fitness routine during the sensor session; continuously measuring lactate concentration with the lactate sensor during the sensor session; storing at least some lactate concentrations measured by the lactate sensor during the sensor session.

Example Embodiment 30, wherein the sensor session lasts at least twelve hours.

Example Embodiment 31, wherein at least two of the plurality of elements of the fitness routine are separated by at least six hours.

Example Embodiment 32, wherein the sensor session lasts at least three days.

Example Embodiment 33, wherein the sensor session lasts at least ten days.

Example Embodiment 34, wherein the lactate sensor is operably connected to sensor electronics, wherein the sensor electronics comprises memory, and wherein the storing comprises storing in the memory of the sensor electronics.

Example Embodiment 35, comprising transmitting stored lactate concentrations to a separate device.

Example Embodiment 36, wherein the separate device comprises a smartphone.

Example Embodiment 37, wherein the lactate sensor is a transcutaneous sensor.

Example Embodiment 38, wherein the lactate sensor is a non-invasive sensor.

Example Embodiment 39, comprising processing a plurality of lactate concentrations measured by the lactate sensor to generate an estimate of aggregate lactate over a period of time.

Example Embodiment 40, wherein the period of time is selected by a user of the lactate sensor.

Example Embodiment 41, wherein the period of time is the duration of the sensor session.

Example Embodiment 42, including a method of sepsis risk monitoring comprising: entering a health care facility; implanting a lactate sensor; undergoing a surgical procedure in the health care facility; leaving the healthcare facility after performance of the surgical procedure with the lactate sensor remaining implanted; leaving the lactate sensor implanted for at least three days after leaving the healthcare facility.

Example Embodiment 43, comprising leaving the lactate sensor implanted for at least ten days after leaving the healthcare facility.

Example Embodiment 44, comprising receiving an indication of sepsis risk from sensor electronics operably coupled to the lactate sensor.

Example Embodiment 45, comprising entering a healthcare facility in response to the indication of sepsis risk.

Example Embodiment 46, wherein the entered healthcare facility is the same healthcare facility where the surgical procedure was performed.

Example Embodiment 47, wherein the surgical procedure is performed on one or more organs of the digestive system.

Example Embodiment 48, wherein the surgical procedure is performed on the esophagus.

Example Embodiment 49, wherein the surgical procedure is performed on the pancreas.

Example Embodiment 50, wherein the subject is at least 60 years old.

Example Embodiment 51, wherein implanting the sensor is performed after entering the healthcare facility.

Example Embodiment 52, wherein implanting the sensor is performed before entering the healthcare facility.

Example Embodiment 53, wherein entering the hospital is performed in accordance with a pre-arranged surgery schedule.

Example Embodiment 54, wherein the lactate sensor is a lactate monitor.

Example Embodiment 55, wherein the lactate monitor comprises sensor electronics.

Example Embodiment 56, additionally comprising affixing a body temperature sensor.

Example Embodiment 57, additionally comprising affixing a heart rate sensor.

Example Embodiment 58, additionally comprising affixing a respiration rate sensor.

Example Embodiment 59, wherein the implanting comprises transcutaneously implanting.

Example Embodiment 60 including an ambulatory analyte monitoring system comprising: an implantable lactate sensor; a body temperature sensor; sensor electronics operably connected to the lactate sensor and the body temperature sensor.

Example Embodiment 61, wherein the sensor electronics is configured to integrate sensor data from the lactate sensor and sensor data from the body temperature sensor to generate a value representative of sepsis risk.

Example Embodiment 62, additionally comprising a heart rate sensor, wherein the sensor electronics is configured to integrate sensor data from the lactate sensor, sensor data from the body temperature sensor, and sensor data from the heart rate sensor to generate the value representative of sepsis risk.

Example Embodiment 63, additionally comprising a respiration rate sensor, wherein the sensor electronics is configured to integrate sensor data from the lactate sensor, sensor data from the body temperature sensor, sensor data from the heart rate sensor, and sensor data from the respiration rate sensor to generate the value representative of sepsis risk.

Example Embodiment 64, comprising a user interface for presenting the value to a subject.

Example Embodiment 65, wherein the value forms a binary output of the system.

Example Embodiment 66, wherein the user interface consists of one or more LEDs that emit one or more colors.

Example Embodiment 67, additionally comprising a display having less than 200 pixels per side.

Example Embodiment 68, additionally comprising a wireless transmitter.

Example Embodiment 69, wherein the system is configured to detect both abnormal body temperature and elevated lactate levels.

Example Embodiment 70, wherein the implantable lactate sensor is transcutaneously implantable.

Example Embodiment 71, including a method of sepsis risk monitoring comprising: implanting a lactate sensor into a patient in the time period between one day before beginning a surgical procedure on a patient and one day after ending the surgical procedure on the patient; leaving the lactate sensor implanted for at least three days after ending the surgical procedure.

Example Embodiment 72, comprising leaving the lactate sensor implanted for at least ten days after ending the surgical procedure.

Example Embodiment 73, wherein the implanting comprises transcutaneously implanting.

Example Embodiment 74, comprising: discharging the patient from the healthcare facility where the surgical procedure was performed; and leaving the lactate sensor installed after the discharge.

Example Embodiment 75, wherein the surgical procedure is performed on one or more organs of the digestive system.

Example Embodiment 76, wherein the surgical procedure is performed on the esophagus.

Example Embodiment 77, wherein the surgical procedure is performed on the pancreas.

Example Embodiment 78, wherein the patient is at least 60 years old.

Example Embodiment 79, including a method of monitoring for sepsis infections comprising: selecting a patient for sepsis monitoring; implanting a lactate sensor into the patient; performing a surgical procedure on the patient; and discharging the patient following the surgical procedure with the lactate sensor remaining implanted.

Example Embodiment 80, wherein the implanting is done before performing the surgical procedure.

Example Embodiment 81, wherein the implanting is done during the surgical procedure.

Example Embodiment 82, wherein the implanting is done after performing the surgical procedure.

Example Embodiment 83, wherein the selecting is done based at least in part on the organs the surgical procedure is directed to.

Example Embodiment 84, wherein the surgical procedure is performed on one or more organs of the digestive system.

Example Embodiment 85, wherein the selecting is done based at least in part on the patient's age.

Example Embodiment 86, including a method of monitoring for post-surgical sepsis infection comprising implanting a lactate sensor within one day of ending a surgical procedure performed in a healthcare facility.

Example Embodiment 87, comprising implanting the lactate sensor after being discharged from the healthcare facility.

Example Embodiment 88, comprising wearing the lactate sensor for at least three days after being discharged from the healthcare facility.

Example Embodiment 89, comprising wearing the lactate sensor for at least ten days after being discharged from the healthcare facility.

What is claimed is:

1. A method of monitoring a patient for sepsis risk, comprising:
measuring, using a lactate monitoring system including a lactate sensor worn by the patient, lactate concentration levels (“lactate concentrations”) associated with the body over one or more time periods; and
identifying, using the lactate monitoring system, a risk of sepsis based on the measured lactate concentrations.

2. The method of claim 1, further comprising:
providing, using the lactate monitoring system, an indication to a user based on the determined risk of sepsis.

3. The method of claim 1, wherein the indication comprises an alert or a notification.

4. The method of claim 1, further comprising:
receiving, at the lactate monitoring system, user input to enter sepsis monitoring mode; and
prior to the identifying, entering, at the lactate monitoring system, the sepsis monitoring mode to monitor the patient for the risk of sepsis, wherein the identifying is based on the lactate monitoring system entering the sepsis monitoring mode.

5. The method of claim 1, wherein:
the one or more time periods at least include a time period subsequent to a sepsis event, and
identifying the risk of sepsis is based on a first set of the lactate concentrations measured during the subsequent to the sepsis event.

6. The method of claim 5, wherein the sepsis event comprises a surgical procedure performed on the patient.

7. The method of claim 5, wherein:
the one or more time periods include at least one time period prior to the sepsis event during which a second set of the lactate concentrations are measured by the lactate sensor; and
identifying the risk of sepsis is further based on the second set of the lactate concentrations.

8. The method of claim 7, wherein the sepsis event comprises a surgical procedure performed on the patient.

9. The method of claim 7, wherein identifying the risk of sepsis is further based on comparing the first set of the lactate concentrations with the second set of the lactate concentrations.

10. The method of claim 7, wherein identifying the risk of sepsis is based on one or more data points derived from the second set of the lactate concentrations.

11. The method of claim 10, wherein:
the one or more data points include a standard deviation associated with the second set of the lactate concentrations,
identifying the risk of sepsis is based on determining that at least one of the first set of the lactate concentrations exceeds an upper bound of the standard deviation.

12. The method of claim 11, wherein the at least one of the first set of the lactate concentrations correspond to a duration of time that exceeds a defined threshold duration of time.

13. The method of claim 12, wherein:
the one or more data points include a baseline lactate concentration derived from the second set of the lactate concentrations,
identifying the risk of sepsis is based on determining that at least one of the first set of the lactate concentrations exceeds the baseline lactate concentration.

14. The method of claim 13, wherein identifying the risk of sepsis is based on determining that the at least one of the first set of the lactate concentrations exceeds a threshold calculated based on the baseline lactate concentration.

15. The method of claim 5, identifying the risk of sepsis is based on determining that one or more of the first set of the lactate concentrations have reached a lactate threshold of 1.3 mmol, 2 mmol, or 4 mmol.

16. The method of claim 5, identifying the risk of sepsis is based on determining that at least a minimum number of the first set of the lactate concentrations is above a lactate threshold of 1.3 mmol, 2 mmol, or 4 mmol.

17. The method of claim 5, wherein identifying the risk of sepsis is based on a rate of change of the first set of lactate concentrations.

18. The method of claim 17, wherein identifying the risk of sepsis is based on at least one of:
the rate of change of the first set of lactate concentrations being lower than a first defined rate of change;
the rate of change of the first set of lactate concentrations persisting for longer than a defined time duration; and
at least some of the first set of lactate concentrations exceeding a defined sepsis threshold for longer than the defined time duration.

19. The method of claim 18, wherein the defined sepsis threshold is a multiple of a baseline lactate concentration derived from the second set of the lactate concentrations.

20. The method of claim 1, further comprising:
using body temperature of the patient over the time period subsequent to the surgical procedure to derive a first body temperature pattern, wherein identifying the risk of sepsis is further based on a deviation of the first body temperature pattern from a second body temperature pattern corresponding to a time period prior to the surgical procedure.

21. The method of claim 20, wherein the using comprises measuring the body temperature over the time period using the lactate monitoring system including a body temperature sensor.

22. The method of claim 1, further comprising:
using body temperature of the patient over the time period subsequent to the surgical procedure, wherein identifying the risk of sepsis is further based on the body temperature exceeding a body temperature threshold.

23. The method of claim 22, wherein the using comprises measuring the body temperature over the time period using the lactate monitoring system including a body temperature sensor.

24. The method of claim 5, further comprising:
using heart rate measurements of the patient over the time period subsequent to the sepsis event, wherein identifying the risk of sepsis is further based on the heart rate measurements indicating an elevated heart rate or a decrease in heart rate variability over the time period.

25. The method of claim 24, wherein the using comprises measuring the patient's heart rate over the time period to generate the heart rate measurements using the lactate monitoring system including a heart rate monitor.

26. The method of claim 5, further comprising:
using respiratory rate measurements of the patient over the time period subsequent to the sepsis event, wherein identifying the risk of sepsis is further based on the respiratory rate measurements indicating an elevated respiratory rate or exceeding a respiratory rate threshold.

27. The method of claim 26, wherein the using comprises measuring respiratory rate of the patient over the time period to generate the respiratory rate measurements using the lactate monitoring system including a respiratory rate monitor.

28. The method of claim 5, wherein identifying the risk of sepsis is further based on determining a likelihood that the first set of the lactate concentrations is indicative of exercise during the time period subsequent to the sepsis event.

29. The method of claim 28, wherein determining the likelihood is based on at least one of heart rate measurements or glucose measurements corresponding to the time period subsequent to the surgical procedure.

30. The method of claim 5, wherein identifying the risk of sepsis is further based on determining a likelihood that first set of the lactate concentrations is indicative of food consumption during the time period subsequent to the surgical procedure.

31. The method of claim 30, wherein determining the likelihood is based on glucose measurements corresponding to the time period subsequent to the surgical procedure.

32. The method of claim 1, further comprising:
upon determining that the determined risk of sepsis corresponds to a first likelihood that the patient has developed sepsis, providing, using the lactate monitoring system, a first indication to a user using a first user interface feature having a first characteristic; and
upon determining that the determined risk of sepsis corresponds to a second likelihood that the patient has developed sepsis, providing, using the lactate monitoring system, a second indication to the user using a second user interface features having a second characteristic.

33. The method of claim 1, wherein the lactate sensor is transcutaneous or non-invasive.
